(19) United States
(12) Patent Application Publication
(10) Pub. No.: US 2018/0135060 A1
(43) Pub. Date: May 17, 2018

(54) ESCHERICHIA COLI T7 EXPRESSION VECTOR, VECTORS FOR THE CO-EXPRESSION AND CO-PURIFICATION OF RECOMBINANT PEPTIDES IN/WITH CARRIER PROTEINS, USE OF EXPRESSION VECTORS FOR OBTAINING COMPLEXES WITH MULTIPLE ANTIGENS AND IMMONOMODULATORS

(71) Applicant: OURO FINO SAUDE ANIMAL LTDA, Distrito Industrial de Cravinhos (BR)

(72) Inventors: Celso Raul Romero Ramos, Ribeirao Preto (BR); Miryam Marroquin Quelopana, Ribeirao Preto (BR)

(73) Assignee: OURO FINO SAUDE ANIMAL LTDA, Distrito Industrial de Cravinhos (BR)

(21) Appl. No.: 15/318,555

(22) PCT Filed: Apr. 20, 2015
(86) PCT No.: PCT/BR2015/050047

§ 371 Date: Dec. 13, 2016

(30) Foreign Application Priority Data
Jun. 13, 2014 (BR) BR1020140145028

(51) Int. Cl. C12N 15/62; C12N 15/70; C12N 15/68; C12N 15/69; C12N 15/65; C07K 14/195; C07K 14/575; C07K 14/30

(52) U.S. Cl. CPC C12N 15/62; C12N 15/70; C12N 15/68; C12N 15/69; A61K 39/00; C07K 14/195; C07K 14/575; C07K 14/30; C12N 15/65

(57) ABSTRACT

The present invention relates to a vector for the expression of recombinant proteins, antigens, pathogen-like particles and immunogenic complexes, said vector (pMRKA vector) being produced by modifying the plasmids containing the gene sequence of the T7 promoter of E. coli, this modification being mainly characterized by the substitution of the ampicillin-resistance gene by the kanamycin-resistance gene, and by the insertion of the par sequence (partition sequence which determines the efficient segregation of the plasmids in daughter cells during cell division). Also provided are expression vectors based on the pMRKA plasmid, which additionally comprise at least one of the gene sequences of the exosome of P. abyssi, which vectors are designated pMRKA-EXO, pMRKA-RING and pSUMAC. The invention also provides the vectors additionally comprising gene sequences with immunomodulatory or immunoregulatory activity, preferably the pMRKA-Z-Z-EXO and pMRKA-Z-Z-RING vectors. Other aspects of the invention include the method for producing said expression vectors and the use of the obtained vectors.

### TECHNICAL FIELD

The present invention refers to a vector for the expression of recombinant proteins herein called pMRKA, comprising a sequence of the T7 bacteriophage promoter of E. coli, kanamycin resistant gene and par sequence (partition sequence which determines the efficient segregation of the plasmids in daughter cells during cell division), in order to increase plasmids stability. Additionally, it is provided expression vectors based on pMRKA plasmids additionally comprising at least one of the P. abyssi exosome gene sequences, such vectors herein called pMRKA-EXO, pMRKA-RING and pSUMAC, that allow the co-expression and co-purification of recombinant proteins, particularly for obtaining complexes with multiples vaccine antigens and immunomodulators.

Other aspects of the invention include a method for producing said expression vectors and the use of the obtained vectors.

### BACKGROUND OF THE INVENTION

Numerous E. coli expression vectors have been developed using strong promoters, the first one used was T7 promoter of T7 bacteriophage. The expression systems based on T7 are broadly used for super-expression in large scale of recombinant proteins in prokaryotes and eukaryotes cells. The system uses polymerase RNA of T7 that is a highly active enzyme, extending RNA chains eight times faster than the RNA polymerase of E. coli.

T7 vectors of E. coli are broadly known, for example, pAE vector was described by Ramos et al. (Ramos et al. 2004, A high-copy T7 Escherichia coli expression vector for the production of recombinant proteins with a minimal N-terminal His-tagged fusion peptide. Braz J Med Biol Res.37(8):1103-9), and having a nucleotides sequence of SEQ ID No:1. pAE vector is a derivative from the commercial vector pRSETA (Life Technologies), from which were taken the sequences of Xpress Epitope, enterokinase cutting site, in order to remain in the modified plasmid only a minimal fusion of non-removable 6xHis. pAE vector is successfully used in bench scale, but its performance is reduced in fermenters with high density cell cultures, because pAE is an unstable plasmid under these fermentation conditions.

Thus, the systems of recombinants proteins expression need to be improved, in order to make possible, among other factors, the recombinant proteins production in industrial scale which require high density cell fermentation; greater number of plasmid copies per cell to assure the desired protein expression; improved stability of the produced proteins; use of selection markers that do not interfere with living organisms, particular animals and humans.

One of the selection markers being used is the ampicillin resistant, antibiotic with broad use in treating human beings and animals. It is obvious that this marker is not desirable due to the consequences that may occur. Hence, this marker has been subject of researches for replacement with another antibiotic not used in humans and animals. For example, document EP1925670 describes E. coli expression vector in which the ampicillin resistant gene was replaced with a kanamycin resistant gene.

Despite the advances already made in the expression vector field, there is still a need for improvements in both the multipurpose plasmids approach and in plasmids oriented for the expression of specific proteins in order to achieve improved conditions for industrial scale production of recombinant proteins.

The vaccines based on monomeric antigens have a low immunogenicity and they cannot provide similar protection as the conventional vaccines, which use whole organisms as attenuated or inactivated bacteria and viruses (Morein B, & Simons K (1985) “Subunit vaccines against enveloped viruses: virosomes, micelles and other protein complexes”. Vaccine. 3:83-93). Due to this, despite the great number of candidate vaccines and publications related to them, there are no vaccines based in this type of antigens available in the market.

Recent studies show that the great molecular mass complexes formation, such as VLPs (Virus Like Particles), Virosomes and ISCOM (antigen-containing lipids vesicles), PLPs (Pathogen Like Particles, obtained by nanotechnology), are highly immunogenic. (Rosenthal J.A. et al. (2014) Pathogen-like particles: biomimetic vaccine carriers engineered at the nanoscale. Current Opinion in Biotechnology (Nanobiotechnology•Systems biology) 28: 51-58). Nevertheless, the process for obtaining such antigens is expensive and complex, thus their use is limited.

Antigen carrier structures with lower complexity were obtained using sequences tending to form fibers and successfully used in laboratory tests: Rudra J S et al. (2010) “A self-assembling peptide acting as an immune adjuvant”. Proc Natl Acad Sci U S A. 107(2):622-7; Rudra J S. et al. (2012) “Self-assembled peptide nanofibers raising durable antibody responses against a malaria epitope”. Biomaterials. 33(27):6476-84; Pepponi I. et al. (2013) Immune-complex mimics as a molecular platform for adjuvant-free vaccine delivery. PLoS One. 8(4):e60855; e Miyata T, et al. (2011) Tricomponent immunopotentiating system as a novel molecular design strategy for malaria vaccine development. Infect Immun. 79:4260-75. In the meantime, the complexes known in the prior art can carry only one antigen and one modulator, and in order to obtain effective protection against bacteria and parasites, it is necessary the use of one more antigen in vaccine formulation.

In the present application, it became possible the use of a archae exosome complex, particularly Pyrococcus abyssi, as a system capable of carrying more than one antigen and immunomodulators, in such a way that is possible co-expressing and co-purifying the antigens from one single culture. As P. abyssi is hyperthermophile, fusion proteins containing the exosome proteins can present thermostability.

### SUMMARY OF THE INVENTION

The objective of the present invention is to provide stable plasmids that allows an enhanced production, in industrial scale, of recombinant proteins both in separated form and as forming multiprotein complexes (these could be thermostable) with application in developing multi antigenic vaccines.

According to the first aspect of the present invention, it is provided a T7 expression vector of Escherichia coli characterized by: (a) having a high number of copies per cell; (b) being highly stable in high density cellular fermentation conditions; (c) sized about 3 Kpb; (d) having an antibiotic resistant marker non-used in humans and animals. More preferably, T7 expression vector of E. coli of the invention is pMRKA plasmid and it comprises a nucleotide sequence SEQ ID No: 19.

According to a second aspect of the invention, there is provided a vector for expressing fusion polypeptides for carrier proteins, having as characteristics: (a) a vector according the first aspect of the invention; and (b) at least one P. abyssi exosome gene encoding at least one immunogenic protein, antigens or immunoregulatory molecules carrier protein. More preferably, said vectors are: pMRKA-EXO plasmid comprising a nucleotide sequence SEQ ID No: 36, pMRKA-RING plasmid comprising a nucleotide sequence SEQ ID NO: 37, and the pSUMAC plasmid comprising the nucleotide sequence SEQ ID NO: 40.

According to a third aspect of the invention, there is provided vectors for expressing fusion polypeptides for carrier proteins, said vectors additionally having immunomodulatory activity and presenting the following characteristics: (a) a vector according to the first aspect of the invention; and (b) at least one P. abyssi exosome gene encoding at least one immunogenic proteins, antigens or immunoregulatory molecules carrier protein and (c) at least one immunomodulation segment, such as the Z domain. More preferably, said vectors are pMRKA-ZZ-EXO plasmid comprising a nucleotide sequence SEQ ID NO: 43, and pMRKA-ZZ-RING plasmid comprising a nucleotide sequence SEQ ID NO: 45.

According to a fourth aspect of the invention, it is provided a method for obtaining an expression vector according to the first aspect of the invention, comprising the following steps: (i) exchange the ampicillin resistant gene in vector of SEQ ID NO: 1 with the kanamycin resistant gene; (ii) amplification of the plasmid fragment of SEQ ID NO: 1 corresponding to the segment located among nucleotides 804 to 1857; (iii) binding the amplified segments from step (ii) with the kanamycin resistant gene; (iv) amplification of the plasmid obtained in step (iii) to insert restriction sites in base 2607 of pMK plasmid; (v) amplification of par sequence and insertion of the amplified sequence resulting among the restriction sites located at the base 2607 from pMK for obtaining pMRK plasmid; and (vi) eliminating the restriction sites of BgIII and Ncol in the kanamycin resistant gene in pMRK vector to obtain pMRKA plasmid.

In a fifth aspect, the present invention refers to a method for obtaining an expression vector according to the second aspect of the invention, comprising the following steps: (i) preparing pMRKA plasmid according to the method of the fourth aspect of the invention; (ii) preparing at least one P. abyssi exosome gene sequence encoding at least one immunogenic proteins, antigens or immunoregulatory molecules carrier protein; (iii) synthesis of one double-stranded DNA containing said at least one P. abyssi exosome gene sequence and (iv) cloning said double-stranded DNA obtained in step (iii) into the said pMRKA plasmid. Such method allows obtaining fusion vectors pMRKA-EXO, pMRKA-RING and pSUMAC.

In a sixth aspect, the present invention refers to a method for obtaining an expression vector according to the third aspect of the invention, comprising the following steps: (i) preparing pMRKA plasmid according to the method of the fourth aspect of the invention; (ii) preparing at least on P. abyssi exosome gene sequence encoding at least one carrying immunogenic proteins, antigens or immunoregulatory molecules carrier protein; (iii) preparing at least one sequence presenting immunomodulatory activity, preferably at least one sequence of Z domain; (iv) synthesis of one double-stranded DNA containing said at least one gene sequence of P. abyssi exosome and at least one P. abyssi exosome and at least one sequence with immunomodulatory activity; and (v) cloning said double-stranded DNA obtained in step (iv) into the said pMRKA plasmid. Such method allows obtaining pMRKA-ZZ-EXO and pMRKA-ZZ-RING fusion vectors.

In a seventh aspect, the present invention refers to the use of vectors according to the first, second and third aspects of the invention, obtained according to the methods from the third, fourth and fifth aspects of the invention on preparing in large scale the thermostable recombinant proteins and additionally with immunomodulatory activity.

### BRIEF DESCRIPTION OF THE DRAWINGS

FIG. 1 shows the modifying sequence flow for obtaining vectors of the present invention: pMRKA, pMRKA-EXO, pMRKA-RING and pSUMAC.

FIG. 2 shows the pAE vector map and fusion sequence (6xHis) and cloning sites, such vector was the plasmid base of modifications which result in the vectors of the invention.

FIG. 3 illustrates the strategy used for exchanging the selection marker (antibiotic) into pAE vector for obtaining pMK vector.

FIG. 4 shows the strategy used for the insertion of par sequence into pMK vector for obtaining pMRK vector.

FIG. 5 shows the analysis of plasmids derived from pAE vector: pmK -pAE vector with KanR marker instead of AmpR; and pMRK—pAE vector with two modifications, kanamycin resistant sequence and par sequence; KAN—corresponding to the amplification products of kanamycin gene, par—amplification products of par sequence of pSC1010 plasmid.

FIG. 6 illustrates the scheme of modifications performed by mutagenesis for obtaining pMRKA plasmid.

FIG. 7 shows the scheme of locus containing PAB0419, PAB0420 and PAB0421 genes which encode Rrp4, Rrp41 and Rrp42 exosome proteins, respectively.

FIG. 8 presents the diagram of Pyrococcus abyssi exosome structure surface; in the middle, the diagram of RNA surface bound to the exosome.

FIG. 9 shows the synthetic gene for in-fusion cloning in the end of N- and C-terminal of Rrp4, Rrp41 and Rrp42 proteins of P. abyssi exosome.

FIG. 10 illustrate a cloning strategy of synthetic gene containing genes of P. abyssi proteins in pMRKA vector.

FIG. 11 shows the co-purification gel from P. abyssi exosome complex protein, co-expressed by pMRKA-EXO plasmid in E. coli.

FIG. 12 illustrates a diagram of exosome core surface of Pyrococcus abyssi, formed by Rrp41 and Rrp42 proteins; in the central region it can be viewed the diagram of the RNA surface bound to the “RING” complex.

FIG. 13 shows a cloning strategy of genes corresponding to the core of P. abyssi exosome in pMRKA vector for obtaining pMRKA-RING.

FIG. 14 shows co-purification of Rrp41 and Rrp42 proteins, co-expressed in pMRKA-RING plasmid, by ion-exchange chromatography. 80° C.—input, material treated at 80° C. for 30 minutes followed by centrifugation; 1-5 fractions corresponding to the elution peak.

FIG. 15 illustrates a diagram of complex surface formed by Rrp42 protein.

FIG. 16 shows the strategy for cloning Rrp42 protein in pMRKA vector for obtaining pSUMAC vector.

FIG. 17 shows purified Rrp42 protein gel in a step of cell lysis at 80° C. for 30 minutes, followed by centrifugation.

FIG. 18 illustrates a strategy for cloning Z-Z domains into pMRKA-EXO plasmid.

FIG. 19 illustrates a strategy for cloning Z-Z domains into pMRKA-RING plasmid.

FIG. 20 illustrates the analysis per SOS-PAGE from the co-purified complex containing P. abyssi exosome proteins with fusion of Z-Z domains in Rrp42 protein. M denotes the Molecular Mass Marker.

FIG. 21 illustrates the analysis per SOS-PAGE from the co-purified complex containing P. abyssi exosome ring (RING) proteins with fusion of Z-Z domains in Rrp42 protein. M denotes the Molecular Mass Marker. 1 -denotes the purified ZZ-RING complex.

FIG. 22 illustrates the strategy for cloning synthetic genes of vaccine antigens of Mycoplasma hyopneumoniaeno in pMRKA-EXO plasmid for obtaining pMRKA-EXOMYC vector.

FIG. 23 illustrates the analysis for SOS-PAGE of polyproteins expression from antigens complexes of Mycoplasma hyopneumoniae with proteins from the P. abyssi exosome. M—denotes Molecular Mass Marker; 1—denotes Rosetta (OE3)/pMRKA-EXOMYC strain; 2 —denotes Rosetta (OE3)/pMRKA-RINGMYC strain; 3—denotes Rosetta (OE3)/pSUMAC-MYC strain; Non-induced denotes culture without the inducer presence; induced—denotes the induced culture with 1 mM ITPG.

FIG. 24 illustrates the result of EXOMYX complex purification in Escherichia coli, by tangential filtration. M . . .—denotes the molecular mass marker; L—denotes he analysis of 2 μL of purified protein; 2—denotes the analysis of 4 μL of purified protein.

FIG. 25 illustrates the strategy for cloning synthetic genes from Inhibin antigen, as described and claimed by the patent application PI102014005376-0, in pSUMAC vector.

FIG. 26 illustrates the analysis for SOS-PAGE of the fusion protein expression Rrp42-IniOF during the formation (induction) and purified product for thermal treatment (100 kDa retained).

### DETAILED DESCRIPTION OF THE INVENTION

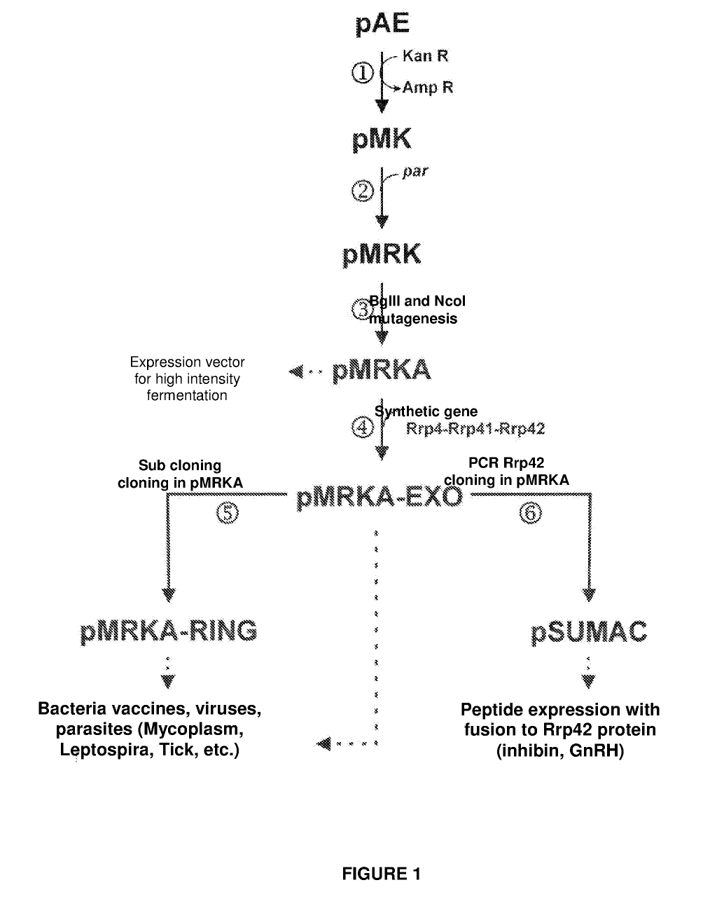

The present invention refers to the obtaining of expression vectors for producing thermostable recombinant proteins, including vectors comprising plasmids for the expression of fusion recombinant proteins with carrier proteins arising from the P. abyssi exosome. More particularly, the invention refers to vectors obtained from pAE plasmids (Ramos et al., 2004), which was obtained by modifying pRSETA commercial plasmid (Life Technologies), mainly by modifying the antibiotic resistant gene (selection marker) and through the insertion of par sequence of pSC101 plasmid. The flowchart of modifications introduced in pAE plasmid and subsequent plasmids resulting from each modification step is represented in FIG. 1.

The term “expression vector” is intended to mean, in the present description, the plasmids in its final form, resulting from the modifications introduced in the initial pAE plasmid and into the subsequent intermediate plasmids.

The terms “immunomodulatory sequence” and “immunoregulatory sequence” are herein used in interchangeably and meaning the sequences that provide immunoregulatory activity to the proteins/polypeptides containing gene sequences with this feature, for example, Z domain from A protein of S. aureus.

The terms “polypeptide” and “recombinant protein” are used herein in interchangeably and meaning amino acid sequences encoded from the plasmids of the invention, said sequences corresponding to recombinant proteins with improved thermostability.

As previously mentioned, pAE vector is adequate for expressing proteins in bench scale, but due to its fermenting instability in high density cultures, its performance becomes unacceptable for producing recombinant proteins in industrial scale. To solve these shortcoming of pAE vector, the following modifications were made according to the invention: (a) substitution of antibiotic resistant gene giving rise to the plasmid herein called pMK; (b) introducing the partition sequence “par” of pSC101 plasmid, resulting in the plasmid herein called pMRK; and (c) eliminating BgIII and Ncol restriction sites from kanamycin resistant gene in pMRK vector.

### pMK Vector

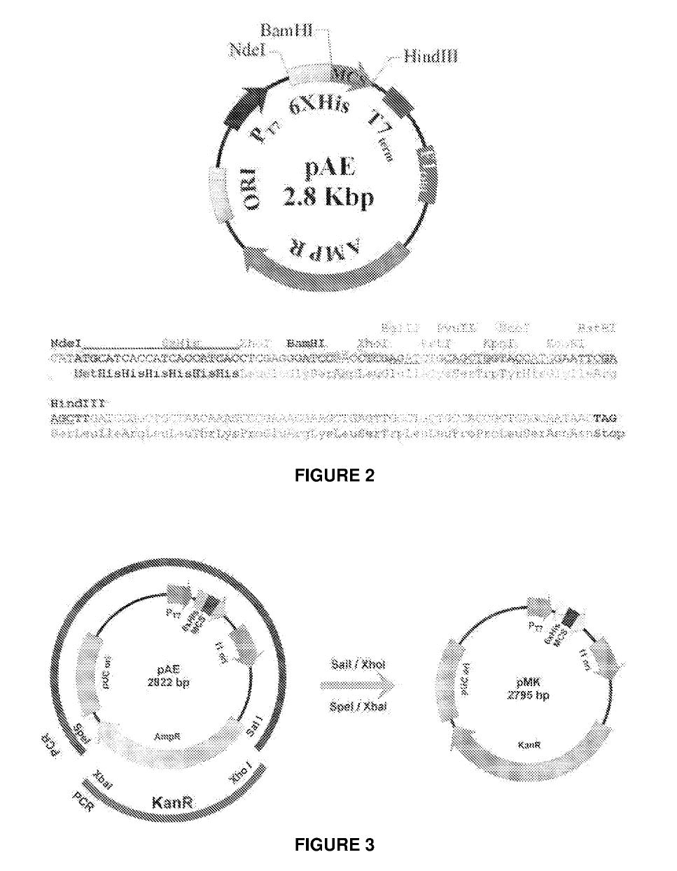

pMK vector results from the substitution of ampicillin resistant gene with kanamycin resistant gene in pAE vector, which comprises the nucleotide sequence SEQ ID NO: 1, and its map is represented in FIG. 2.

The need to modify the pAE plasmid was due to the following reasons: (a) as the ampicillin resistant gene encodes a beta-lactamase which is located in periplasmic space of E. coli in high density culture, this enzyme has the possibility to migrate of the culture means and, consequently, to degrade the antibiotic, decreasing the selective pressure of the means and providing the buildup of cells without plasmid; and (b) in the production of expression vectors in industrial scale it is not recommended to use antibiotic similar to the ones used in medical and veterinary fields, as it is the case of ampicillin. As kanamycin does not have medical application, the ampicillin resistant gene in pAE vector was replaced with kanamycin resistant gene.

To achieve such replacement of antibiotic resistant gene, it was made a modification into the skeleton of pAE vector using “whole plasmid PCR” technique, which allows operating precise exchanges, as represented in the scheme showed in FIG. 3.

According to the invention, it was used kanamycin resistant gene of pK18 plasmid (access GenBank number M17626.1 [Pridmore R D 1987. New and versatile cloning vectors with kanamycin-resistance marker. Gene. 56(2-3):309-12]). Such plasmid contains the transponson gene Tn5 that encodes a neomycin phosphotransferase enzyme (NPT), which provides resistance both to a neomycin (neo) and as well as kanamycin (kan). For the amplification, by PCR, of the region that encodes NPT together with its promoter (of the nucleotide 208 to nucleotide 1182, in the plasmid sequence pK18 (M17626.1 GenBank No.) the following primers were designed:

Kan-For

SEQ ID No: 2

ATCTCGAGTT ATGGACAGCAAGCGAACC
3′Kan-Rev

SEQ ID No: 3′

CATCTAGAATTTCGAACCCCAGAGTCC 3′

Kan-For primer contains the restriction site Xhol and Kan-Rev primer the Xbal site (both underlined in the primer sequences). PCR was made with a high fidelity enzyme KOO DNA polymerase (Novagen) and used as mold the pK18 plasmid. As a result, the sequence containing kanamycin resistant gene (SEQ ID No: 4) was amplified, and the resulting gene encodes the NPT enzyme (SEQ ID No: 5).

Additionally, it was amplified, by PCR, a pAE plasmid fragment, from nucleotide 804 to nucleotide 1857, which does not contain ampicillin resistant gene. This amplification was made using the primers:

VecMar-For

SEQ ID No: 6

5′ CGACTAGTGCATTGGT AACTGTCAGACC 3′
VecMar-Rev

SEQ ID No: 7

5′ ATGTCGACGTGCCACCT AAATTGTAAGCG 3′

The VecMar-For primer contains the restriction site Spel, and the VecMar-Ver primer contains the restriction site SaII (both underlined in the primer sequences) in order to provide action of the enzymes that do not have cutting sites in pAE sequence. The amplification was achieved using a high fidelity enzyme KOO XL DNA polymerase (Novagen), indicated for amplifying DNA extensive fragments, as integral plasmids.

The cuts with SaII enzymes, in the amplified DNA of pAE vector, and Xhol in amplified DNA of kanamycin resistant gene, generate compatible ends. Such fact allows the gathering of these DNAs, at the same time wherein, in case the original cutting sites are lost, it is not necessary the introduction of cutting sites into the new vector during its construction. This also happens with Spel enzymes (in the amplified segment of the vector) and Xbal (in the amplified segment of KanR) that generate compatible ends after cut (see FIG. 3). The binding of the vector amplified segments (cut with enzymes SaII and SpeI and from KanR (cut with Xhol and Xba, result on plasmid called pMK, selected by its kanamycin resistance (see FIG. 3) (SEQ ID No: 8)).

### DMRK Vector

Despite improved by the substitution of the antibiotic resistant gene, pMK vector still presents unsatisfying stability, which was overcome, according to the present invention, by the input of a sequence, known as “par”, that has the attribute to enhance plasmids stability.

In order to increase the stability of the plasmids of the invention, it was used the strategy of introducing the partition sequence “par” (that determines the efficient segregation of plasmids in the daughter cells during cell division) of pSC101 plasmid in pAE vector. The use of par sequence to enhance plasmid stability was previously described by Meacock et al. (Meacock PA and Cohen S N (1980) Partitioning of bacterial plasmids during cell division: a cis-acting locus that accomplishes stable plasmid inheritance. Cell. 20(2):529-42; WO 1984001172 A 1). It is worth mentioning that, although this strategy is very interesting under the point of view of stability of the obtained plasmids, currently, the systems broadly disseminated in research field for example, pET vectors) and in industrial scale production, they do not include the use of “par” sequence.

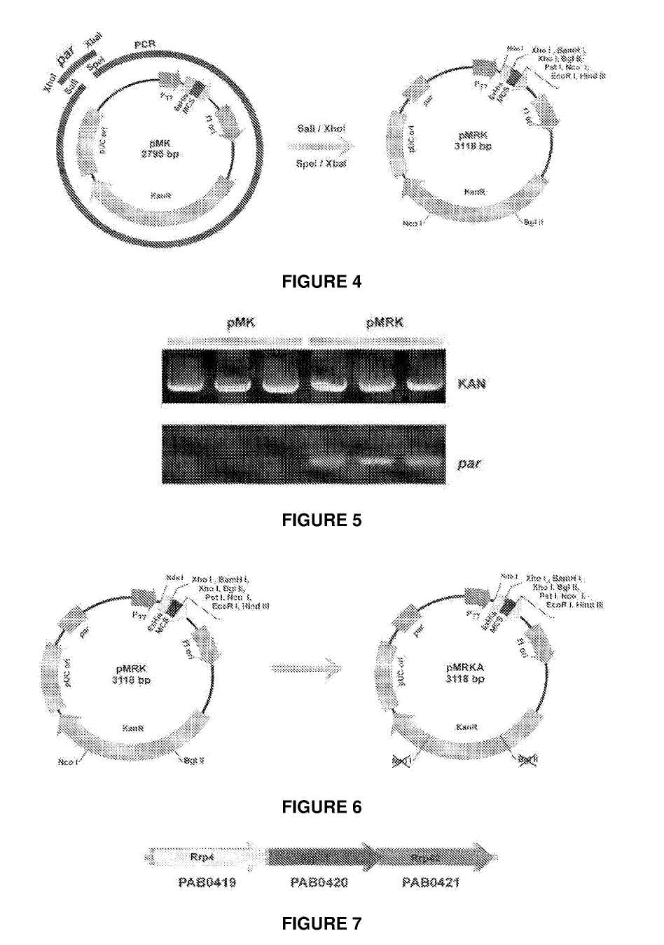

Thus, to increase stability of plasmids based on pAE vector, the partition sequence “par” was input in pMK plasmid using, one more time, a “whole plasmid PCR” technique, as shown in FIG. 4.

In pMRK plasmid diagram, shown in FIG. 4, it is highlighted the position of the cutting sites for restriction enzymes BgIII and Ncol, both in selection marker KanR, as in cloning multiple site.

As source of “par” sequence, it was used the pSC101 plasmid itself (GenBank Access N° NC_002056, [Yamaguchi &Masamune Y. 1985. Autogenous regulation of synthesis of the replication protein in plasmid pSC101. Mal Gen Genet. 200(3):362-7]). To amplify the par sequence (from the nucleotide 4605 to nucleotide 4876, of GenBank sequence NC_002056) and subsequent insertion of such sequence in pMK vector, the following primers were designed:

ParFor

SEQ ID No: 9

3′ ATCTCGAGTTTGTCTCCGACCATCAGG 3′
ParRev

SEQ ID No: 10

5′ GTTCTAGACGGGAT AATCCGAAGTGG 3′

ParFor primer contains the restriction site Xbal, and Par-Rev primer contains the site for Xhol (both underlined in the primers sequence). PCR was made with a high fidelity enzyme KOO DNA polymerase (Novagen) and used as mold the pSC101 plasmid. As a result of the amplification, it was obtained the “par” sequence (SEQ ID NO: 11).

Additionally, pMK plasmid was amplified by PCR to input restriction sites in the base position 2607 from this plasmid, thus providing the insertion of “par” sequence amplified segment, through the use of the following primers:

VecPar-For

SEQ ID No: 12

5′ ATACTAGTACGCGGCCTTTTTACGGTTCC 3′
VecPar-Rev

SEQ ID No: 13

5′ ATGTCGACTGCTGGCGTTTTTCCAT AGG 3′

The VecMar-For primer contains Spel restriction site, and the VecPar-Ver primer contains SaII site (both underlined in the primer sequences) in order to provide action of the enzymes that do not have cutting sites in pMK sequence. The amplification was achieved using a high fidelity enzyme KOO XL DNA polymerase (Novagen), which is indicated for amplifying whole plasmids.

As it was previously described, cuts with SaII and Xhol enzymes generate compatible ends of the digested DNA, in the same way as the cuts with Spel and Xbal enzymes. The binding of the vector amplified segments (cut with enzymes SaII and SpeI and “par” sequence (cut with Xhol and Xba, resulted in the plasmid called pMRK, (SEQ ID NO:14)) (see FIG. 4) selected by its kanamycin resistance.

FIG. 5 shows the analysis by PCR of pMK and pMRK plasmids in relation to the presence of kanamycin resistant marker and par sequence.

### pMRKA Vector

Finally, in order to eliminate restriction BgIII and Ncol sites in kanamycin resistant gene, i.e., in KanR marker from pMRK vector, it was made two site-directed mutagenesis, using the PCR technique and using the “Quick Change Site-Directed Mutagenesis Kit” (Stratagene), as shown in FIG. 6.

Firstly, it was made the mutagenesis in BgIII site. This site is located after the promoter region of the gene which encodes neomycin phosphotransferase (NPT) enzyme, that provides kanamycin (kan) resistance. To effect C976T mutation, that corresponds to cytosine from site (AGATCT), BgIFor and BgIRev primer were designed, whose sequences are shown as follows:

BglFor

(SEQ ID No: 15)

GATGGCGCAGGGGATCAAGATTTGATCAAGAGACAGGATGAG
BglRev

(SEQ ID No: 16)

CTCATCCTGTCTCTTGATCAAATCTTGATCCCCTGCGCCATC

The position of the nucleotides that form BgIII site is underlined and in the table is outstanding the mutagenic nucleotide. After mutagenesis cycle by PCR using “QuikChange Site-Directed Mutagenesis Kit” (Stratagene), the resulting plasmids were characterized by restriction analysis with BgIII enzyme and par sequencing with Kan-For primer (SEQ ID No: 2) in order to verify if the mutagenesis was successful.

Subsequently, it was made the mutagenesis on Ncol site. This site is located on the region that encodes kanamycin resistance (kanR). To make the neutral mutation T1571C in the Thymine from the site (CCAIGG), that corresponds to a third position in encoding CAT codon of His188 in Neomycin Phosphotransferase, the primers NcoFor and NcoRev were designed. The alteration from CAT to CAC does not change this amino acid residue once the CAC codon keeps encoding Histidine.

NcoFor

SEQ ID No: 17

ATCTCGTCGTGACCCACGGCGATGCCTGCTTGC
NcoRev

SEQ ID No: 18

GCAAGCAGGCATCGCCGTGGGTCACGACGAGAT

The position of the nucleotides that form Ncol site is underlined and it is highlighted, in the table, the mutagenic nucleotide. After mutagenesis cycle by PCR using “QuikChange Site-Directed Mutagenesis Kit” (Stratagene), the resulting plasmids were characterized by restriction analysis with Ncol enzyme and par sequencing with Kan-Rev primer (SEQ ID No: 3) in order to verify if the mutagenesis was successful.

After the two mutagenesis cycle, BgIII and NcoI sites, in cloning multiple sites, became single sites into the plasmid sequence, and thus they could be used for the desired cloning. The plasmid without cleavage sites BgIII and Ncol in kanamycin resistant gene was named pMRKA (SEQ ID No: 19) and it corresponds to the first aspect of the present invention. Such vector encodes 6xHis fusion protein (SEQ ID No: 20) and NPT enzyme, with neutral mutation T1571C (SEQ ID No: 21).

The stability of pMRKA in industrial scale fermentation culture (increased cell density) was successfully tested.

pMRKA plasmid corresponds to the vector of the first aspect of the invention and it represents an advantageous option in relation to pET vector, which is the commercial vector used in most processes for producing recombinant proteins. The advantages of pMRKA are as follows:
Smaller size: 2,8 Kpb against 5.4 kbp of pET; thus, pMRKA can carry bigger sized sequences.
Higher number of copies per cell: pMRKA vector has 300-500 copies per cell against 20-50 copies of pET per cell; thereby pMRKA has a bigger gene charge and, in some cases, bigger expression compared with pET vector. pMRKA also facilitates the experiments of cloning and sequencing.
Greater stability: the presence of “par” sequence makes pMRKA more stable in relation to pET plasmid.

### pMRKA-EXO Vector

The advantages of pMRKA above mentioned allow using this plasmid for constructing a derivative vector expressing fusion proteins with carrier proteins, such vector comprises sequences encoding Rrp4, Rrp41 and Rrp42 proteins of P. abyssi proven important as antigens or immunogenic complexes-carrying proteins.

The bacteria Pyrococcus abyssi is a hyperthermophile archaebacterium, which lives near the hydrothermal vents on the ocean floor. This archae tolerates temperatures above 100° C. and a pressure up to 200 atmospheres, however, it does not represent a health hazard for human beings, animals or plants. To tolerate such high temperatures and pressures, the evolution made P. abyssi to have extremely stable proteins, resistant to denaturation by temperature or pressure.

The genes that encode the proteins composing exosome complex of Pyrococcus abyssi are located in a single locus in genome of such archaebacteria (Koonin E V et al. (2001) Prediction of the archaeal exosome and its connections with the proteasome and the translation and transcription machineries by a comparative-genomic approach. Genome Res. 11:240-252) (see FIG. 7).

In the process of the present invention, the exosome complex of P. abyssi was assembled in vivo and in vitro using recombinant proteins expressed in Escherichia coli. This complex was previously functionally and structurally described (Ramos C R, et al. (2006) The Pyrococcus exosome complex: structural and functional characterization. J Biol Chem. 281 :6751-6759; e Navarro M V et al. 2008. Insights into the mechanism of progressive RNA degradation by the archaeal exosome. J Biol Chem. 283:14120-14131).

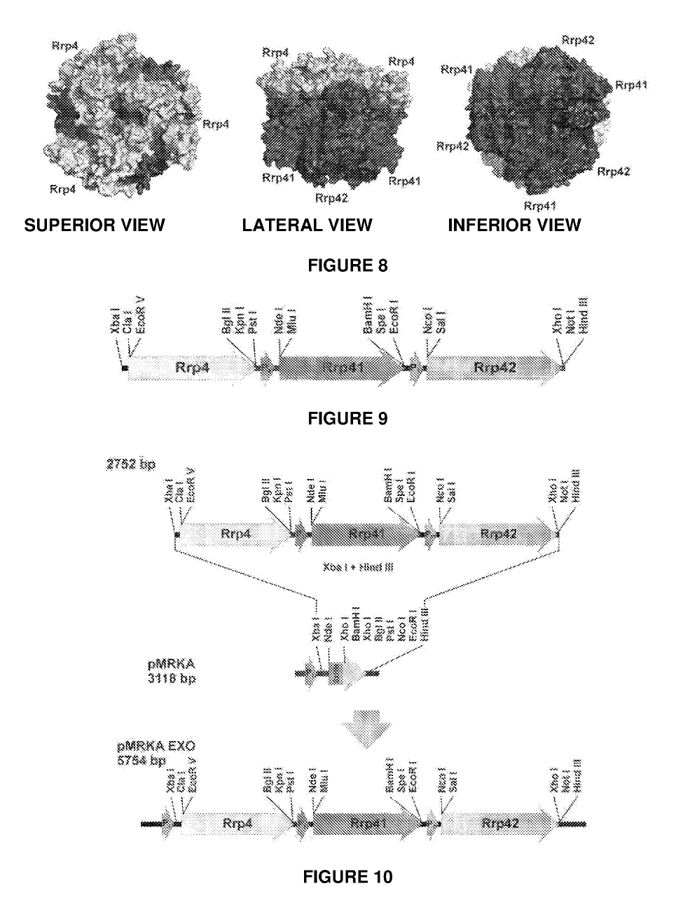

In FIG. 8, it is shown surface models of exosome structure of P. abyssi. The structure of the complex named “RNases PH ring” formed by Rrp41 and Rrp42 was determined by crystallography (Navarro et al. 2008), while the structure and position of Rrp4 was modeled using as mold the archae exosomes structures Archaeoglobus fulgidus (Buttner K et al. (2005) Structural framework for the mechanism of archaeal exosomes in RNA processing. Mal Cell. 20:461-471) and Sulfolobus solfataricus (Lorentzen E, & Conti E. Crystal structure of a 9-subunit archaeal exosome in pre-catalytic states of the phosphorolytic reaction. Archaea. 2012:721869).

On the structure base of the exosome, it can be visualized a hexameric ring (“RING”) formed by three copies of Rrp41 and Rrp42 proteins. This ring has binding activity to the RNA ad catalytic activity (RNase). Above the ring there are located three copies of Rrp4 protein, which contains binding domains to RNA.

This compact structure is highly soluble and stable, and can be expressed in high level in Escherichia coli, such characteristics facilitate its obtaining.

### The Exosome as Carrier of Vaccine Antigens

The archae exosome complex has unique characteristics that could be explored on developing new generation vaccines named “pathogen like particles”, which use biomimetic to enhance immunologic response, such as:
Capability of specific self-assembly when said complex is expressed in cells of Escherichia coli.
High expression, solubility and thermostability of the complex, which facilitates the purification and formulation of recombinant proteins.

This complex also provides higher stability to the immunogenic protein during formulation and the storage of completed products, important aspect on developing biological products.

Ability to carry more than one antigen and immunomodulatory protein because, according to the structure determined by crystallography, the amino ends and terminal carboxyl from the three proteins are exposed to the solvent.

The technique of the present invention, using gene sequences of the P. abyssi exosome, represents a great advance in relation to the use of recombinant antigens alone in vaccine formulation, these antigens that cannot provide enough protection level as the level that is reached with whole organism as inactivated bacteria and viruses (Rosenthal J.A. et al. (2014) Pathogen-like particles: biomimetic vaccine carriers engineered at the nanoscale. Current Opinion in Biotechnology (Nanobiotechnology•Systems biology) 28: 51-58).

The possibility of gathering in a complex more than one antigen and immunomodulators make the proposed system of the present invention be better than the strategies reported using sequences tending to form fibers such as Rudra J S et al. (2010) “A self-assembling peptide acting as an immune adjuvant”. Proc Natl Acad Sci U S A. 107(2):622-7; Rudra J S. et al. (2012) “Self-assembled peptide nanofibers raising durable antibody responses against a malaria epitope”. Biomaterials. 33(27):6476-84; Pepponi I. et al. (2013) Immune-complex mimics as a molecular platform for adjuvant-free vaccine delivery. PLoS One. 8(4):e60855; and, Miyata T, et al. (2011) Tricomponent immunopotentiating system as a novel molecular design strategy for malaria vaccine development. Infect Immun. 79:4260-75.

### Synthetic Gene for Expression of P. abyssi Exosome

To explore the possibility of using the exosome as a multiple protein carrying complex, according to the invention, it was designed a DNA sequence containing genes for Rrp4, Rrp41 and Rrp42 proteins of Pyrococcus abyssi (in this order, as they are found in genome of P. abyssi, see FIG. 7), with restriction sites that allow in-fusion cloning of recombinant proteins in N- or C-terminal ends of exosome components.

FIG. 9 shows projected sequence scheme according to the invention.

The construction shown in FIG. 9 is planned for the cloning in pMRKA vector between Xbal and HindIII sites. Thereby, only the first gene (Rrp4) stays under control of T7 promoter of the vector. For genes Rrp41 and Rrp42 it was designed sequences containing T7 promoter, in similar design to the co-expression vectors pETDuet-1 and pACYDuet-1 (EMO Millipore), but without the operating sequence of operon lac, present in these commercial vectors.

### Sequences Among Encoding Regions of Exosome Proteins

The first fragment design contains, from the 5′ end, the cutting site for Xbal enzyme, the sequence transcribed from T7 promoter of pMRKA vector, the ribosome binding site (Ribosome Binding Site (RBS)), the translation start codon (highlighted in the square) and restriction sites Clal and EcoRV for N-terminal in-fusion cloning with Rrp4 protein, as shown below.

Rrp4 RBS

SEQ ID NO: 22

XbaI RBS Clar EcoRV
TCTAGAAATAATTTTGTTTAACTTTAAGAAGGAGATATATATATGGCTAT
CGATGCGGATATCGCC M A I D A D I A

The second designed fragment contains the cutting site for BgIII enzyme, aiming C-terminal in-fusion cloning of Rrp4 protein, stop codon and Kpnl and Pstl sites for unidirectional cloning. It follows the following sequences: a sequence containing T7 promoter for Rrp41 protein, the ribosome binding site, the translation start codon (highlighted in the square) and restriction sites Ndel and Mlul for N-terminal fusion cloning with Rrp41 protein, as shown below.

Rrp4 end and Promotor of Rrp41 and start

SEQ ID NO: 23

BglII KpnI PstI
AGATCTTAAGGTACCCTGCAGGCTTAAGTCGAACAGAAAGTAATCGTATT
GTACACGGCCGCAACT
R S * Rrp4
T7 promoter
TCGAAATCAATACGACTCACTATAGGGAGACCACAACGGTTTCCCATCTT
AGTATATTAGTTTAAC RBS NdeI MluI
TTTAAGAAGGAGATATACATATGGCTACGCGTGCA M A T R
A Rrp41

The third designed fragment contains the cutting site for BamHl enzyme for C-terminal in-fusion cloning with Rrp41 protein, stop codon and Spel and EcoRl sites, for unidirectional cloning. Continuing, it follows the following sequences: a sequence containing T7 promoter for Rrp42 protein, the ribosome binding site, the translation start codon (highlighted in the square) and restriction sites Ncol and Sall for N-terminal in-fusion cloning with Rrp42 protein: Rrp41 end and Promoter for Rrp42 and start -SEQ ID NO: 24

BamHI SpeI EcoRI
GGATCCTAAACTAGTGAATTCGCTTAAGTCGAACAGAAAGTAATCGTATT
GTACACGGCCGCATA G S * Rrp41
T7 Promoter
ATCGAAATTAATACGACTCACTATAGGGAGACCACAACGGTTTCCCATCT
TAGTATATTAGTTAA
RBS NcoI SalI GTATAAGAAGGAGATATACCATGGGTGTCGAC
M G V D Rrp42

At last, the fourth designed fragment contains the cutting site for Xhol enzyme for C-terminal in-fusion cloning with Rrp42 protein, the stop codon, and Natl and HindIII sites (in 3′ end), for unidirectional cloning.

Rrp42 end

(SEQ ID NO: 25)

XhoI NotI HindIII CTCGAGTAAGCGGCCGCAAGCTT
L E * Rrp42

### Design of Synthetic Genes for Rrp4. Rrp41 and Rrp42 Proteins

For designing synthetic genes, it was used the strategy based on the theory known as “Codon Harmonization” (Angov E. et al. (2011) Adjustment of codon usage frequencies by codon harmonization improves protein expression and folding. Methods Mol Biol. 705:1-13). According the theory of codon harmonization, the rare codons (that are less used than other codons in a given organism) have lower amount of tRNAs and perform an important role on protein folding. They are located in protein loops, among the defined secondary structure segments. These codons could decrease the protein synthesis speed through the ribosome, allowing the folding of the synthetized sequence before the beginning of a synthesis of another protein fragment.

The rare codons on PAB0419, PAB0420 and PAB0421 genes, that encodes Rrp4, Rrp41 and Rrp42 proteins, respectively, were identified through the use of ANACONDA program (http://bioinformatics.ua.pt/software/anaconda) feed with the usage of P. abyssi codons table, obtained from the database “Codon Usage Database” (http://www.kazusa.or.jp/codon/).

The localization of encoded amino acids by “rare codons” in tridimensional structures obtained for each protein was made using Swiss-Pdb-Viewer 4.0.1 program (http://spdbv.vitaHt.ch). In the “harmonization” of the synthetic gene, it was considered the rare codons that encode amino acids located in loops regions among secondary structure sequences, alpha-helix and beta-sheet.

For designing the synthetic gene, it was used Gene Designer 2.0 program (DNA2.0), manually selecting “rare” codons” of E. coli for each one of the amino acids identified by ANACONDA program and located in the structure by Swiss-Pdb-Viewe 4.0.1, in view to enhance protein folding. The Gene Designer 2.0 program was feed with the table called Usage of Codons for Highly Expressed Proteins in E.coli (Villalobos et al. (2006) GeneDesigner: a synthetic biology tool for constructing artificial DNA segments. BMC Bioinformatics. 7:285).

The amino acid sequences used for designing synthetic genes correspond to those related in GenBank for Pyrococcus abyssi strain GES:

TABLE 1
Gene sequences for P. abyssi used in the present invention

| | | | Considered | | Encoded |
|---|---|---|---|---|---|
| Protein | GenBank | No aa | for synthesis | Synthetic Gene | protein |
| Rrp4 | NP 126300 | 265 | 2-262 | SEQ ID No: 26 | SEQ ID |
| | | | | | No: 27 |
| Rrp1 | NP 126301.1 | 249 | 3-246 | SEQ ID No: 28 | SEQ ID |
| | | | | | No: 29 |
| Rrp42 | NP 126302.1 | 274 | 2-274 | SEQ ID No: 30 | SEQ ID |
| | | | | | No: 31 |

In the drawings it was not considered the proteins starting methionine (e.g., Rrp41 has two methionines in the beginning of the translation).

Rrp41 protein contains catalytic site responsible for the RNase activity of the exosome. As this activity is not desired for the developing of vaccines, the present amino acids in active site were replaced, based on published results (Navarro M V et al. 2008. Insights into the mechanism of progressive RNA degradation by the archaeal exosome. J Biol Chem. 283:14120-14131). In this paper it is mentioned that the double mutation R89E and K91E in Rrp41 abolished the RNase activity of the exosome. In the synthetic gene it was made exchanges with R89T and K91S that resulted in a smaller change in the molecule charge compared with mutations reported in the literature.

Thus, the Rrp4 binding activity (that contains the binding domains to RNA S1 and KH) to ssRNA was maintained, as well as the ring structure (RING) formed by Rrp41-Rrp42 proteins. Without being limited to a specific theory, it is believed that the ssRNA of E. coli, that is co-purified with the exosome, can active TLR7 receptor and, thus, act as an adjuvant in exosome based vaccines.

At last, the sequences were gathered in the following order: SEQ ID No:22-SEQ ID No: 26-SEQ ID No: 23-SEQ ID No: 28-SEQ ID No: 24-SEQ ID No: 30-SEQ ID No: 25, to obtain a complete sequence corresponding to the diagram in FIG. 10 -SEQ ID NO: 32.

The Rrp4, Rrp41 and Rrp42 protein sequences, with modifications in amino ends and carboxyl terminal contained in sequence SEQ ID No: 32 are presented in sequences SEQ ID No: 33, SEQ ID No: 34 and SEQ ID No: 35, respectively.

Additionally, a double-stranded DNA containing the sequence SEQ ID No: 32 was synthetized and cloned by Xbal and HindIII sites in pMRKA vector, as shown in diagram in FIG. 10, resulting in pMRKA-EXO vector of the present invention.

According to the method of the fourth aspect of the invention, for the construction of pMRKA-EXO vector, pMRKA vector is the preferred on for being stable, with greater number of copies per cell, when compared with the vector form pET series and, furthermore, because it has a smaller size, which facilitates cloning of other genes in fusion with exosome proteins of P. abyssi. pMRKA-EXO plasmid comprises SEQ ID No: 36.

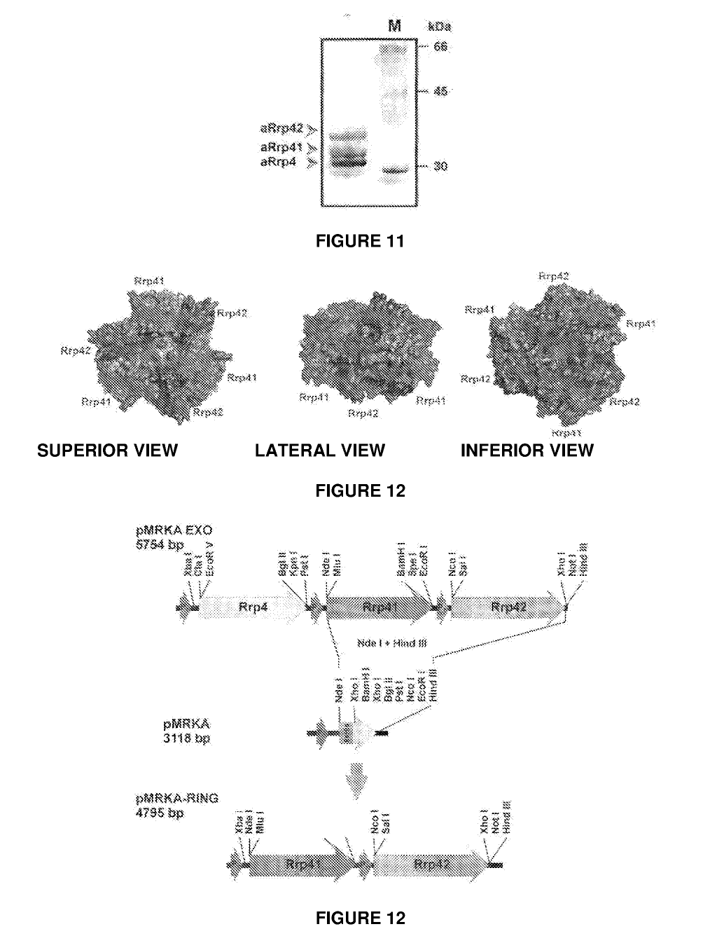

FIG. 11 shows the result of the co-purification of Rrp4, Rrp41 and Rrp42 proteins, expressed in Escherichia coli by pMRKA-EXO plasmid.

### pMRKA-RING Vector

### Ring of Exosome RNases PH of P. abyssi

The core of the exosome is constituted by the hexamer formed by Rrp41 and Rrp41 proteins. Such structure in gel form is known as “RNases PH ring” and is herein called “RING” (see FIG. 12).

This structure, simpler than the whole exosome structure in FIG. 8, can also be used to fuse antigens and immunoregulatory molecules in the “pathogens like particles” concept as previously described. The RNase activity was abolished before R89T and K91S mutations in Rrp41 protein, while the binding activity of one-stranded RNA (ssRNA) was maintained. It is believed that ssRNA of E. coli, which co-purified together with the “RING” (already previously detected in crystalline structure of this complex (Navarro et al. 2008)) can activate the TLR receptor and, thus, act as an adjuvant, improving the immunologic response of vaccines based on this complex.

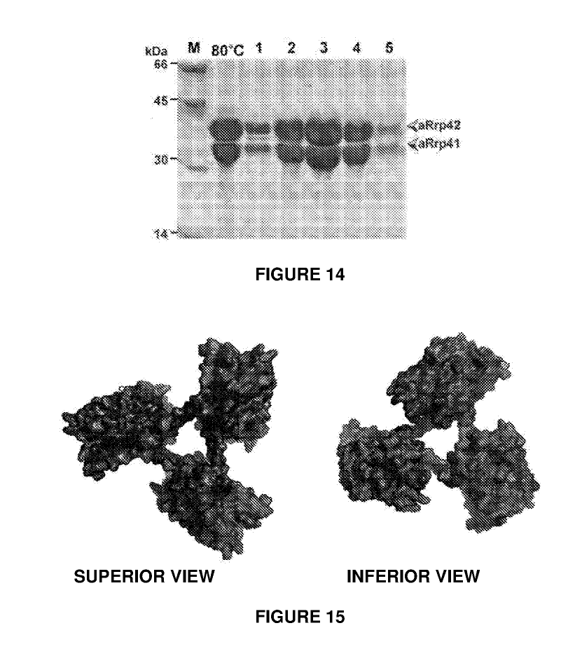

FIG. 13 shows a cloning strategy for obtaining the vector according to the second aspect of the present invention, which contains Rrp41 and Rrp42 proteins of P. abyssi, and it is herein named pMRKA-RING, that comprises the sequence SEQ ID No: 37. FIG. 14 shows the result of Rrp41 and Rrp42 proteins co-purification, expressed in E. coli transformed with pMRKA-RING plasmid of the present invention.

### SUMAC Vector

During the individual characterization of exosome proteins of P. abyssi, it was verified that Rrp42 was more soluble and thermostable when compared with Rrp4 and Rrp41 proteins. The analysis of this protein by gel-filtration shows a spin radius corresponding to a trimeric structure, as can be viewed on structural model in FIG. 15.

In the same way as the commercial vectors, such as MBP, GST and SUMO, for the fusion of recombinant proteins, these characteristics of Rrp42 protein (high expression, solubility, thermostability and multimerization) are extremely helpful when producing a protein, e.g., fusion immunogenic protein for increase the recombinant proteins solubility.

Rrp42 protein was tested by means of BepiPred 1.0 program (www.cbs.dtu.dk/services/BepiPred), during the development of recombinant vaccines, it has been proved that its greater immunogenicity compared with other molecules used as peptides and proteins carrier, such as ovalbumin, BSA, chloramphenicol acetyltransferase (CAT), among others. In other words, besides facilitating purification of this type of fusions, Rrp42 provides a greater immunogenicity than the recombinant protein carrier commercially available.

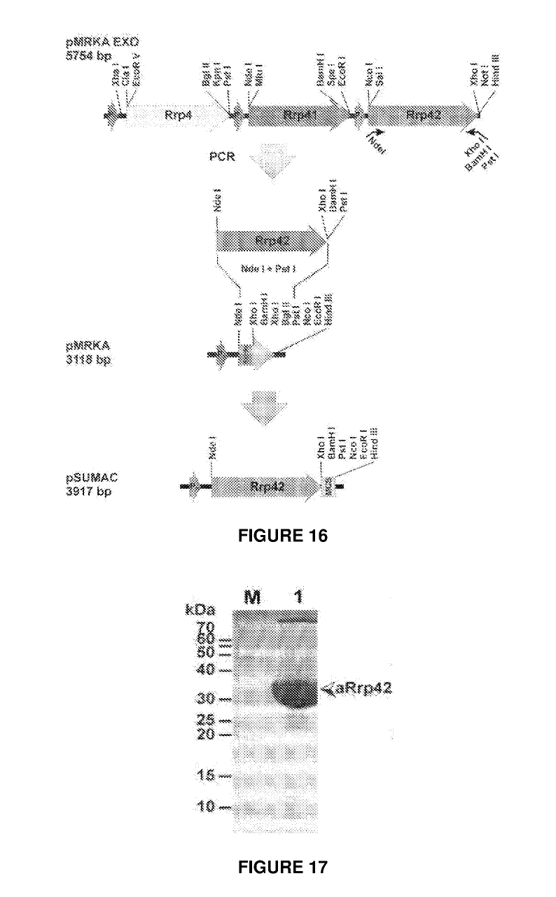

Thus, according to an aspect of the invention, it was constructed a vector for in-fusion expression with Rrp42 protein. FIG. 16 shows the cloning strategy used to reach this purpose.

The sequence corresponding to Rrp42 protein was amplified by PCR from pMRKA-EXO plasmid, using the following nucleotides:

P42-For

SEQ ID No: 38

Nde I
ACCATATGGGTTCTGATAATGAAATCGTG
P42-Rev

SEQ ID No: 39)

PstI BamHI XhoI
AACTGCAGGGATCCCTCGAGACCACCCTGTTTGGCCTTCTCAACAGC

The sequences for cutting sites for Ndol, Pstl, BamHl and Xhol restriction enzymes were underlined. The amplified DNA fragment was digested with Ndeol and Pstl enzymes and clones within the corresponding sites in pMRKA vector, as shown in FIG. 16. Thereby, it was obtained, according to the second aspect of the invention, pSUMAC vector for expressing recombinant proteins in fusion with Rrp42 protein, such vector comprises SEQ ID No: 40.

FIG. 17 shows the result of thermal lysis of E. coli cells transformed with pSUMAC plasmid.

As can be verified from the previous description, pMRKA and a T7 expression vector of E. coli, with high number of copies per cell is highly stable in fermenting conditions with high cellular density.

This vector, which corresponds to the first aspect of the invention, can be used for expressing recombinant proteins with or without N-terminal fusion of 6xHis tail. Additionally, according to the second aspect of the invention, pMRKA-EXO, pMRKA-RING and pSUMAC vectors are vectors derived from pMRKA for expressing proteins of C- or N-terminal fusion with exosome components of Pyrococcus abyssi.

Briefly, pMRKA-EXO vector comprises pMRKA vector and Rrp4-Rrp41-Rp42 protein encoding gene sequences of Pyrococcus abyssi exosome; pMRKA-RING vector comprises pMRKA vector and Rrp41-Rp42 protein encoding gene sequences of Pyrococcus abyssi exosome, and pSUMAC vector comprises pMRKA vector and Rp42 protein encoding gene sequence of Pyrococcus abyssi exosome.

According to the second aspect of the invention, pMRKA-EXO and pMRKA-RING could be used for co-expressing and purification several antigens at the same time, fused to the exosome components, intending to form pathogens like particles.

Still according to the second aspect of the invention, pSUMAC plasmid could be used as peptide vaccines carrier, providing greater solubility and thermostability to such vaccines and facilitating purification by thermal denaturing of host proteins (E. coli). Such characteristic is unique among fusion and carrier proteins currently used, facilitating the production of recombinant proteins in industrial scale with low cost.

Additionally, fuse plasmids with archae exosome proteins, i.e., pMRKA-EXO, pMRKA-RING and pSUMAC, provide greater stability for the produced recombinant proteins, both in formulation and in storing the completed product containing them.

According to the third aspect of the invention, there are provided pMRKA plasmids derivatives that besides comprising fusion of such plasmid to P. abyssi carrier proteins, they also include at least one sequence with immunomodulatory activity, particularly at least one Z domain sequence, which provides to the resulting polypeptides complexes, characteristics of immunomodulation of antigenic activity of said complexes. Z domains is a derivative from the A protein immunoglobulins binding domain of Staphylococcus aureus (Ljungberg, et al. (1993) “The interaction between different domains of staphylococcal protein A and human polyclonal IgG, IgA, IgM and F(ab)2: separation of affinity from specificity”. Mal. Immunol. 30:1279-1285). This domain has the ability to stimulate an immune response (Bekeredjian-Ding et al. (2007) “Staphylococcus aureus Protein A Triggers T Cell-Independent B Cell Proliferation by Sensitizing B Cells for TLR2 Ligands”. J Immuno1.178:2803-2812), such characteristic can be used for driving the interaction of chimeric antigens with cells presenting antigens, in order to increase vaccines immune response. Recently, Miyata et al. (Miyata et al., 2011, “Tricomponent immunopotentiating system as a novel molecular design strategy for malaria vaccine development”. Infect Immun. 79:4260-75) showed that the efficacy of the complex containing a malaria antigen, Z domain and a polymerization sequence, wherein Z domain has a fundamental role for inducing a immunoprotective response.

Miyata et al. (2011) report the difficulty of processing the obtained complex due to the inclusive corpuscle formation that require denaturation and refolding to obtain a soluble complex. Contrary to the results obtained by these researchers, the complexes obtained in the present invention are soluble and can be easily purified.

Besides Z domain, there are other immunogenic domains that could be used in the present invention to provide immunomodulatory activity to the polyproteins complexes from the vectors of the present invention.

As follows, there are provided examples that illustrate and represent specific embodiments of the invention. The following examples should not be understood as limiting the present invention.

### EXAMPLES

### Example 1

Preparing the inoculum:

Unfreezing the aliquot of producer strain and inoculation in LB-kanamycin medium.

Subsequently, the mixture is cultured and stirred in Shaker, in 1L erlenmeyer with 200 ml of LB-KAN (25 pg/ml). Culture at 37° C., 200 rpm for 14-16 hours (DO_{600 nm} from 1 to 2).

Fermentation:

The obtained culture in the previous step is transferred to a vat filled with 5 L of the medium complex high density (MCHD). After reaching DO₆₀₀ₙₘ of 20, it is started the induction for feeding with Lactose, until complete for 22 hours of culture.

Cell Collection:

Cells are collected by centrifugation in 6000 rpm for 30 minutes, in temperature of 8° C. in 1 L bottles.

Cell Lysis:

The cells are resuspended in buffer (5 ml per gram of pellet): 30 mM Tris-HCI pH 9,0; 0,1% Triton X-100;; 5 mM 2-mercaptoethanol.

Afterwards, the cells are treated for 1 hour at 90° C., in hot water bath, homogenizing after each ten minutes in Turrax homogenizer (5000 rpm).

Lysate Clarification:

The lysed and homogenized cells are subjected to filtration through a 0.2 μm membrane in room temperature.

Treatment with Benzonase Enzyme:

The filtered material is treated with benzonase enzyme for 8 hours and 37° C. temperature in order to eliminate contaminating nucleic acids.

100 kDa Membrane Filtering

The withdrawal of digested nucleic acid and benzonase debris and final conditioning of the recombinant protein in the storing buffer made through diafiltration in the tangential flow filtering system, through a 100 kDa hollow membrane.

Sterile Filtration Through a 0.2 μm Membrane

At last, it is executed a filtration in sterile environment through a 0.2 pm membrane. The produced batch is stored in sterile form until its use at 4° C.

The fermentation process described in this Example 1 corresponds to a general methodology that could be used on preparing and purifying polyproteins and polyprotein complexes obtained from vectors of the present invention.

### Example 2

### Fusion of Z-Z Domains from A Protein of Staphylococcus aureus in Pyrococcus exosome Rrp42 Protein

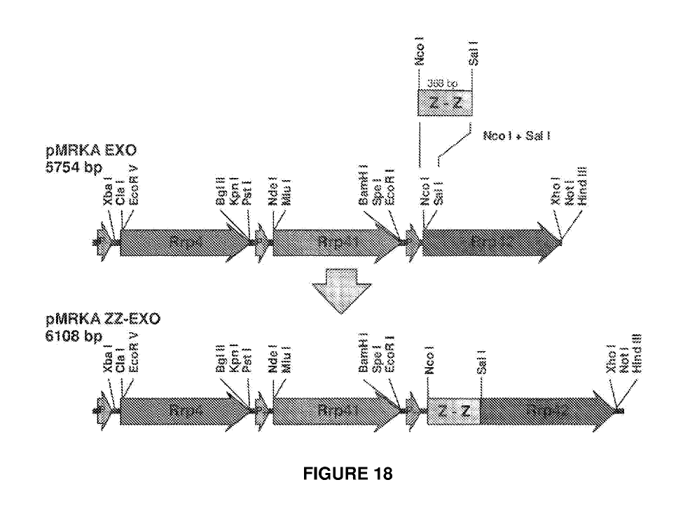

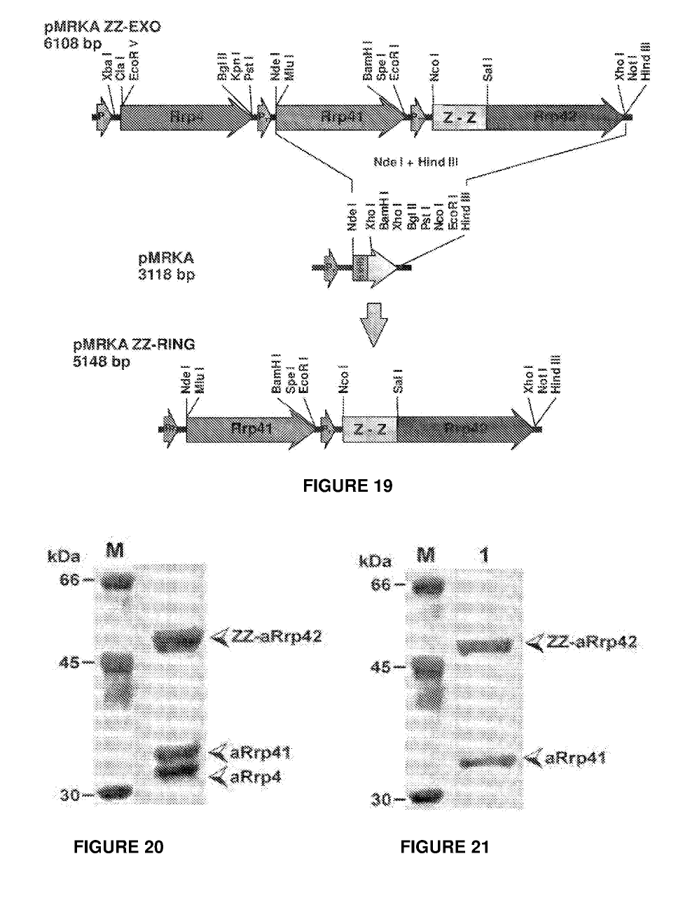

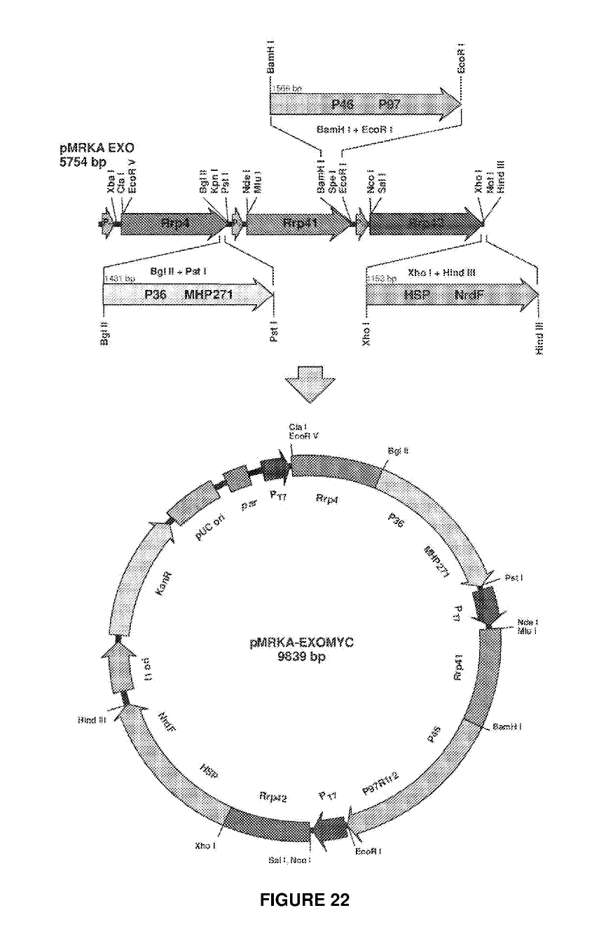

The immunomodulatory activity of Z domain together with exosome protein complex, that in the present invention is used as antigen carrier, was verified by means of a gene synthesis (SEQ ID No: 41), that encodes an amino acid sequence through the expression, in tandem, of two Z-domain copies, herein named Z-Z domains (SEQ ID No: 42). The nucleotide sequence (SEQ ID N: 41) was in-fusion cloned in region corresponding to an Rrp42 protein N-terminal end, using Ncol and Sall sites in pMRKA-EXO and pMRKA-RING plasmids. FIGS. 18 and 19 present representative diagrams of this strategy, resulting in pMRKA-ZZ-EXO (SEQ ID No: 43) and pMRKA-ZZ-RING (SEQ ID No:45)) plasmids, respectively, and consequently in Rrp42 protein expression with Z-Z domain fusion in its N-terminal end (SEQ ID No: 44).

To verify if the ZZ domains fusion interferes with thermostability and purification of the complexes related to Pyrococcus abyssi exosome, Rosetta (DE3) competent cells were transformed with pMRKA-ZZ-EXO and pMRKA-ZZ-RING plasmids. After the transformation, it was made a recombinant protein induction experiment. In the end of such induction, the cells were recovered, resuspended in 300 mM Tris-HCI buffer pH 8.0, and lysed by high pressure homogenization (105 kPa (1000 Bar)). The homogenized was incubated at 80° C. for 30 minutes and cooled in ice for 5 minutes before centrifuging for 30 minutes at 15000 xg. The clarified and thermally treated lysate was charged in a chromatographic column packed with Q-sepharose XL resin and balanced with lysis buffer (30 mM Tris-HCI pH 8.0). After column washing with the same buffer, the bind protein was eluted with NaCI gradient (0-500 mM) in the same buffer. FIGS. 20 and 21 show the purification result of ZZ-EXOSOME and ZZ-RING complexes, respectively.

The results shown in FIGS. 20 and 21 indicate that the fusion with Z-Z domains does not interfere either with the solubility or stability of whole exosome complexes and exosome RING (RING), and that it is possible to obtain them with high level of purity with only a single chromatographic step.

In each complex, there are three copies of Rrp42 protein (see FIG. 8 and FIG. 12), therefore, there are also three copies of Z-Z domain, resulting in an amount of 6 Z-domains per complex, which could facilitate the interaction of these complexes with antigens presenting cells. Further, C-terminal ends are free of exosome proteins to fuse antigens, what represents greater ability than the complex described in Miyata et al. Therefore, pMRKA-ZZ-EXO (SEQ ID No: 43) and pMRKA-ZZ-RING (SEQ ID No: 45) plasmids could also be used to charge antigens in part of the exosome and from the exosome core ring, respectively, together with Z-Z domains.

### Example 3

### Obtaining Protein Complexes Containing Mycoplasma hyopneumiae Antigens

Mycoplasma hyopneumoniae is one of the main pathogenic agents that affects pig breeding. Various vaccine candidates have already been identified, however, none of them, separately, can induce protection against disease in the same level as the commercial bacteria. Aiming to use the exosome complex to charge several antigens with Mycoplasma hypneumoniae, three chimeric proteins encoding genes were synthetized: 36-MHP271; P46-P97R1 R2 and HSP7O-NrdF, corresponding to vaccine antigen pairs. The synthetic genes were clone in pMRKA-EXO vector by BgIII-Kpnl; BamHl-EcoRl and XhoI-HindIII sites, respectively.

The resulting pMRKA-EXOMYC plasmid can express the following polyproteins:
Rrp4-P36-MHP271 (80.5 kDa)
Rrp41-P46-P97R1 R2 (82.7 kDa)
Rrp42-HSP-NrdF (72.6 kDa).

In similar way, P46-P97R1 R2 and HSP7O-NrdF chimeric proteins genes were cloned in pMRKA-RING vector by BamHI-EcoRl and XhoI-HindIII sites, respectively, obtaining pMRKA-RINGMYC plasmid which express the polyproteins:
Rrp41-P46-P97R1 R2 (82.7 kDa)
Rrp42-HSP-NrdF (72.6 kDa).

At last the HSP7O-NrdF chimeric protein gene was cloned in pSUMAC vector by Xhol-HindIII sites, obtaining pSUMAC-MYC plasmid which express the polyprotein:
(a) Rrp42-HSP-NrdF (72.6 kDa).

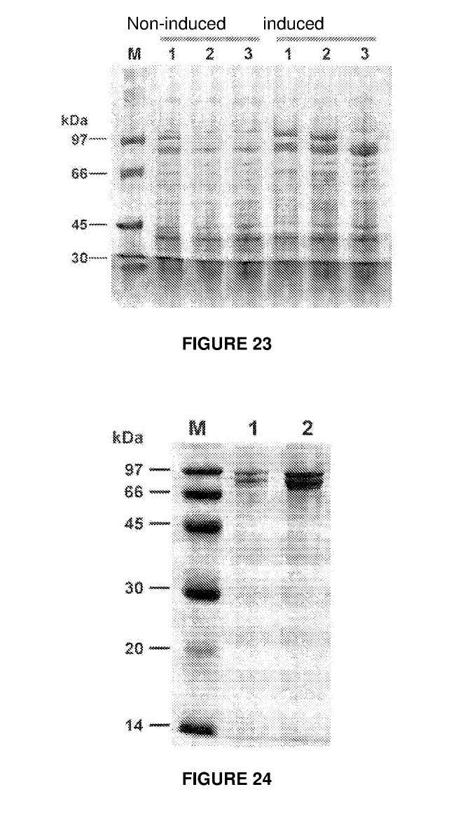

The Rosetta (DE3) competent cells were transformed with pMRKA-EXOMYC, pMRKA-RINGMYC, pSUMAC-MYC plasmids. After transformation, an induction experiment was made to visualize the induced ribbon in each transforming strain. FIG. 23 shows the result of this experiment.

The result in FIG. 23 confirms polyprotein expression through vectors of the present invention, including co-expression of 3 polyproteins in one single vector, a fact without precedent in the prior art. The induced ribbon that migrate among markers 66 and 97 corresponds to the Rrp42-P216A

HSP-NrdF protein (expressed by pMRK-Rrp42-EXO plasmids compared with strip 3 in FIG. 23). It is also possible to conclude, by the comparison of pMRK-EXOMYC (strip 1 in FIG. 23) and by pMRK-RING-MYC (strip 2 in FIG. 23) induced proteins, that there is a migration overlapping in ribbons corresponding to Rrp4-P216B-P36-MHP271 (80.5 kDa) and Rrp41-P46-P97R1 R2 (82.7 kDa) polyproteins into SOS-PAGE gel.

### Example 4

### Purification of Protein Complexes Containing Mycoplasma hyopneumiae Antigens

BL21 Escherichia coli (DE3) strain transformed with pMRK-EXOMYC plasmid was added in 200 ml of LB medium containing kanamycin (25 μg/ml). The culture was incubated at 37° C., 200 rpm for 16 hours.

The 200 ml culture was inoculated in 5L of MCHD (Medium Complex High Density) in 15 liter fermenter. The fermentation was made batch fed constant regimen, at 37° C., 30% dissolved oxygen, having glycerol as carbon source. In DO₆₀₀ₙₘ=15, the feeding by glycerol was changed with lactose (induction) and the fermentation continued for more 12 hours at 28° C.

The cells were collected by 6000 rpm centrifuging for 30 minutes and resuspended in lyse buffer (50 mM Tris-HCI, pH 8.0; 0.1% Triton X-100; 0.25% Sarcozyl) and lysed in high pressure homogenizer.

The lysed and homogenized cells were subjected to filtering through a 0.2 μm membrane (end-filtration Millipore system) at room temperature.

MgCl₂ was added up to a 5 mM final concentration and 90 μm Benzonase (final concentration), and the mixture was incubated at room temperature for 6 hours.

The material treated with Benzonase was concentrated and diafiltered in the tangential filtration system using a 500 kDa hollow fiber membrane of MWCO, in 20 mM Tris-HCI, pH 8,0; 100mM NaCI and 0.1mM EDTA buffer. The retained protein is sterilized by micro filtering with a 0.2 μm membrane and stored at 4° C. FIG. 24 shows the result of polyproteins co-purification which composes a Exomyc complex. The antibody formation induction against each one of the antigens of M. hyopneumoniae included in the complex (data not shown) confirm the usefulness of P. abyssi exosome complex as great complexity antigen carrier.

### Example 5

### Purification of Rrp42-Inhibin Fusion Protein

The vaccination with inhibin is a promising technique in the field of animal fertilization, as well as for increasing egg production in poultry. In the prior art, a number of studies about this application can be found. This vaccine would prevent the use of hormone that are more difficult and expensive to produce and formulate (see Findlay et I. (1993) “Inhibin as a fecundity vaccine”. Animal Reproduction Science. 33: 325-343.

The most used antigen for this type of vaccine is a peptide containing the first 29 amino acids of inhibin alpha subunit conjugated with carrier proteins to stimulate immune response. Nevertheless, the immune response is low. To improve the response, it is used great amounts of fusion antigen, reaching amounts of 1 to 5 mg per dose, and application of up to 5 doses (see Wang et al. (2009) “The long-term effect of active immunization against inhibin in goats”. Theriogenology. 71: 318-22).

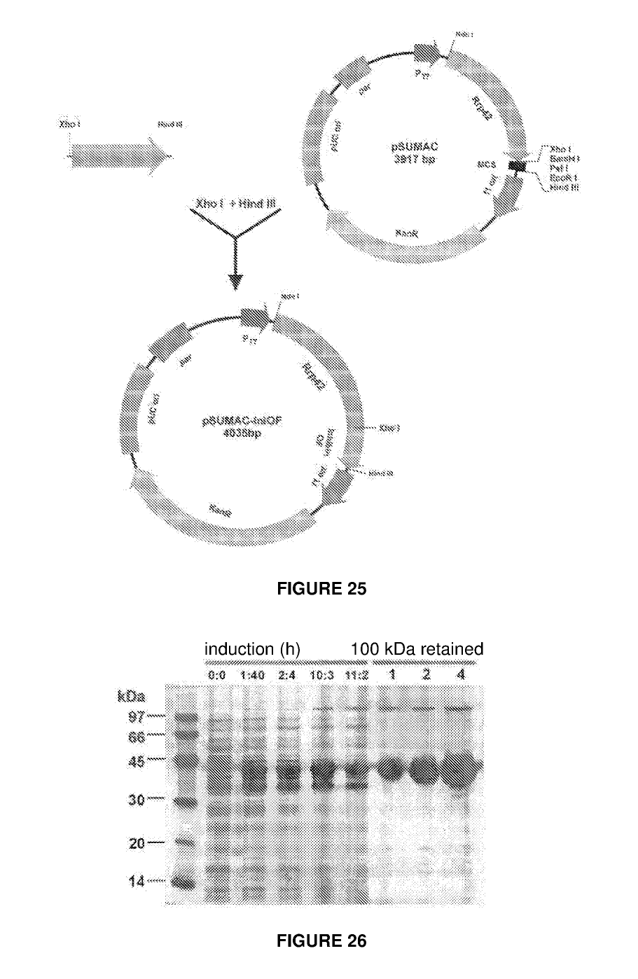

The present invention made possible to design and synthetize a gene which encodes a 49 amino acids proteins containing more alpha-inhibin immunogenic regions. This sequence was clones by Xhol and HindIII sites in pSUMAC vector, as shown in FIG. 25. The protein containing more immunogenic regions of inhibin and its obtaining are detailed in co-pending patent application P1102014005376-0.

BL21 Escherichia coli (DE3) strain transformed with pSUMAC-IniOF plasmid was added in 200 ml of LB medium containing kanamycin (25 pg/ml). The culture was incubated at 37° C., 200 rpm for 16 hours. The culture was used to inoculate 5 L of MCHD (Medium Complex High Density) for executing the fermentation in the same way as in the previous example.

After the fermentation, the cells were collected by 6000 rpm centrifuging for 30 minutes, at 8° C. in 1 liter bottles and resuspended in 30mM Tris-HCI pH 8.0; 0.1% Triton X-100 buffer. The cells were incubated for 1 hour at 90° C., in hot water bath, homogenizing after each ten minutes in Turrax homogenizer (5000 rpm).

The thermally lysed cells were subjected to filtration through a 0.2 μm membrane at room temperature, to clarify the lysate. The filtered material is treated with benzonase enzyme for 8 hours and 37° C. temperature in order to eliminate contaminating nucleic acids. The withdrawal of digested nucleic acid and benzonase debris for final conditioning of the recombinant protein in the storing buffer was made through diafiltration in the tangential flow filtering system, through a 100 kDa hollow membrane.

FIG. 26 shows the result of the fermentation and final purified product using only tangential filtration.

The results shown indicate that fusion with Pyrococcus abyssi exosome proteins, despite its complexity, results in proteins soluble in Escherichia coli cytoplasm. In some cases, these fusion proteins are also thermo resistant, which can be used for purifying recombinant proteins in a simple manner, by thermal treatment for removing contaminant proteins arising from E. coli, a microorganism that is mesothermal.

We claim:

1. A T7 expression vector of Escherichia coli comprising a pMRKA plasmid having the nucleotide sequence SEQ ID No: 19.

2. The Vector of claim 1, having 300 to 500 copies per cell; being highly stable in high density cellular fermentation conditions; sized about 2 Kpb to 4kpb; and having an antibiotic resistant marker.

3. (canceled)

4. The Vector of claim 2, wherein the stability in high density cellular fermentation conditions is provided by the presence of a par sequence of pSC101 plasmid.

5. The Vector of claim 4, wherein the par sequence comprises SEQ ID No: 11.

6. The Vector of claim 2, wherein said antibiotic resistant marker is the kanamycin resistant gene.

7. The vector according to claim comprising at least one P. abyssi exosome gene encoding at least one immunogenic protein, antigen or immunoregulatory molecules carrier protein.

8. The Vector of claim 7, comprising at least one P. abyssi exosome gene encoding at least one of Rrp4, Rrp41 and Rrp42 proteins.

9. (canceled)

10. (canceled)

11. The Vector of claim 8, wherein the vector is selected from the group consisting of:
the pMRKA-EXO plasmid comprising the nucleotide sequence SEQ ID No: 36;
the pMRKA-RING plasmid comprising the nucleotide sequence SEQ ID No: 37; and
the pSUMAC plasmid comprising the nucleotide sequence SEQ ID No: 40 .

12. (canceled)

13. (canceled)

14. (canceled)

15. (canceled)

16. The Vector of claim 7 additionally comprising at least one immunoregulatory gene sequence.

17. (canceled)

18. (canceled)

19. (canceled)

20. (canceled)

21. A Method for obtaining an expression vector, the method comprising the steps: (i) exchange of an ampicillin resistant gene in a vector of SEQ ID NO: 1 with a kanamycin resistant gene; (ii) amplification of the plasmid fragment of SEQ ID NO: 1 corresponding to the segment located among nucleotides 804 to 1857; (iii) binding the amplified segments from step (ii) with the kanamycin resistant gene; (iv) amplification of the plasmid obtained in step (iii) to insert restriction sites in base 2607 of pMK plasmid; (v) amplification of par sequence and insertion of the amplified sequence resulting among the restriction sites located at the base 2607 from pMK for obtaining pMRK plasmid; and (vi) eliminating the BgIII and NcoI restriction sites in the kanamycin resistant gene in pMRK vector for obtaining pMRKA plasmid.

22. The Method of claim 21, wherein said resistance gene exchange of step (i) is executed by amplifying the segment obtained from primer of SEQ ID No: 2 and SEQ ID No: 3, and the amplified segment comprises the SEQ ID No: 4.

23. The Method of claim 21, wherein said amplification of step (ii) is executed from primers of SEQ ID No: 6 and SEQ ID No: 7.

24. The Method of claim 21, wherein said binding step (iii) results in pMK plasmid that comprises SEQ ID No: 8.

25. The Method of claim 21, wherein said par sequence amplification of step (v) is executed from primers of SEQ ID No: 9 and SEQ ID No: 10 resulting in the amplified sequence SEQ ID No: 11.

26. (canceled)

27. The Method of claim 21, wherein said elimination of BgIII and NcoI restriction sites in kanamycin resistant gene of step (vi) is executed by site-directed mutagenesis, and the elimination of said BgIII restriction site is executed using primers of SEQ ID No: 15 and SEQ ID No: 16, and the elimination of said Ncol restriction site is executed using primers of SEQ ID No: 17 and SEQ ID No: 18.

28. (canceled)

29. The Method for obtaining an expression vector according to claim 21, additionally comprising, the steps of:
(i) preparing at least one P. abyssi exosome gene sequence encoding at least on immunogenic proteins, antigens or immunoregulatory molecules carrier proteins; (ii) synthesis of a double-stranded DNA containing said at least one P. abyssi exosome gene sequence; and iii cloning said double-stranded DNA obtained in step (ii) in said pMRKA plasmid.

30.-47. (canceled)

48. A method of expression of recombinant proteins, antigens or immunogenic complexes comprising use of a T7 expression vector of Escherichia coli comprising a pMRKA plasmid having the nucleotide sequence SEQ ID No: 19.

49. (canceled)

50. The method of claim 48, wherein the method is used in expression of recombinant proteins, antigens or immunogenic complexes fused to carrier proteins.

51. The method of claim 50, wherein said carrier proteins are P. abyssi exosome proteins, selected from the group consisting of Rrp4, Rrp41 and Rrp42 proteins.

52. The method of claim 51, wherein the vector is a plasmid selected from the group consisting of pMRKA-EXO, pMRKA-RING, and pSUMAC.

53.-54. (canceled)


SEQUENCE LISTING (45 sequences)

SEQ ID NO: 1 - DNA, 2822 bp, Artificial Sequence
  Sequence of T7 expression pAE vector
  primer_binding (825)..(843): primer binding to VecMar-Rev
  primer_binding (1818)..(1837): primer binding to VecMar-For
gatctcgatcccgcgaaattaatacgactcactatagggagaccacaacggtttccctctagaaataattttgtttaactttaagaaggagatatacatatgcatcaccatcaccatcacctcgagggatccgacctcgagatctgcagctggtaccatggaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 2 - DNA, 28 bp, Artificial Sequence
  Forward primer of kanamycin gene for PCR
atctcgagttatggacagcaagcgaacc

SEQ ID NO: 3 - DNA, 27 bp, Artificial Sequence
  Reverse primer of kanamycin gene for PCR
catctagaatttcgaaccccagagtcc

SEQ ID NO: 4 - DNA, 990 bp, Escherichia coli
  CDS (165)..(959)
atctcgagttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgtctagatg

SEQ ID NO: 5 - protein, 264 aa, Escherichia coli [sequence omitted]

SEQ ID NO: 6 - DNA, 28 bp, Artificial Sequence
  Forward primer for PCR amplification of pAE vector without the gene AmpR
cgactagtgcattggtaactgtcagacc

SEQ ID NO: 7 - DNA, 29 bp, Artificial Sequence
  Reverse primer for PCR amplification of pAE vector without the gene AmpR
atgtcgacgtgccacctaaattgtaagcg

SEQ ID NO: 8 - DNA, 2835 bp, Artificial Sequence
  pAE-derived vector with marker KanR instead of AmpR
  primer_binding (2590)..(2609): PCR VecPar-Rev primer binding
  primer_binding (2610)..(2630): PCR VecPar-For primer binding
gatctcgatcccgcgaaattaatacgactcactatagggagaccacaacggtttccctctagaaataattttgtttaactttaagaaggagatatacatatgcatcaccatcaccatcacctcgagggatccgacctcgagatctgcagctggtaccatggaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 9 - DNA, 27 bp, Artificial Sequence
  Forward primer for PCR amplification of par sequence of plasmid pSC101
atctcgagtttgtctccgaccatcagg

SEQ ID NO: 10 - DNA, 26 bp, Artificial Sequence
  Reverse primer for PCR amplification of par sequence of plasmid pSC101
gttctagacgggataatccgaagtgg

SEQ ID NO: 11 - DNA, 287 bp, Artificial Sequence
  Amplified par sequence of plasmid pSC101
atctcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagaac

SEQ ID NO: 12 - DNA, 29 bp, Artificial Sequence
  Forward primer for PCR amplification of plasmid pMK for insertion of par sequence
atactagtacgcggcctttttacggttcc

SEQ ID NO: 13 - DNA, 28 bp, Artificial Sequence
  Reverse primer for PCR amplification of plasmid pMK for insertion of par sequence
atgtcgactgctggcgtttttccatagg

SEQ ID NO: 14 - DNA, 3118 bp, Artificial Sequence
  pAE-derived vector with the marker KanR and the insert of par sequence
gatctcgatcccgcgaaattaatacgactcactatagggagaccacaacggtttccctctagaaataattttgtttaactttaagaaggagatatacatatgcatcaccatcaccatcacctcgagggatccgacctcgagatctgcagctggtaccatggaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 15 - DNA, 42 bp, Artificial Sequence
  Forward oligonucleotide for C976T mutagenisis in the restriction site BglII of KanR gene
gatggcgcaggggatcaagatttgatcaagagacaggatgag

SEQ ID NO: 16 - DNA, 42 bp, Artificial Sequence
  Reverse oligonucleotide for C976T mutagenisis in the restriction site BglII of gene KanR
ctcatcctgtctcttgatcaaatcttgatcccctgcgccatc

SEQ ID NO: 17 - DNA, 33 bp, Artificial Sequence
  Forward oligonucleotide for T1571C mutagenisis in the restriction site NcoI of gene KanR
atctcgtcgtgacccacggcgatgcctgcttgc

SEQ ID NO: 18 - DNA, 33 bp, Artificial Sequence
  Reverse oligonucleotide for T1571C mutagenisis in the restriction site NcoI of gene KanR
gcaagcaggcatcgccgtgggtcacgacgagat

SEQ ID NO: 19 - DNA, 3118 bp, Artificial Sequence
  pMRK-derived vector with double mutation C976T and T1571C, eliminating the restriction sites BglII and NcoI, respectively, in the gene KanR
  promotor (20)..(39): T7 promotor
  CDS (100)..(240): Fusio hexe histidine
  misc_feature (103)..(110): 6xHis tag
  misc_feature (121)..(173): Multiple cloning sites
  terminator (189)..(313): T7 transcription termination
  primer_binding (223)..(242): T7 Reverse primer
  rep_origin (384)..(839): Origin of Phage f1
  promotor (858)..(930): Promotor of Neomycin Fosfotransferase (gene KanR)
  CDS (1008)..(1802): Gene for Neomycin Fosfotransferase (gene KanR)
  rep_origin (1986)..(2941): Origin of pUC
  misc_feature (2611)..(2689): par sequence
gatctcgatcccgcgaaattaatacgactcactatagggagaccacaacggtttccctctagaaataattttgtttaactttaagaaggagatatacatatgcatcaccatcaccatcacctcgagggatccgacctcgagatctgcagctggtaccatggaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 20 - protein, 46 aa, Artificial Sequence
  Synthetic Construct
MHHHHHHLEGSDLEICSWYHGIRSLIRLLTKPERKLSWLLPPLSNN

SEQ ID NO: 21 - protein, 264 aa, Artificial Sequence [sequence omitted]

SEQ ID NO: 22 - DNA, 66 bp, Artificial Sequence
  Sequence for introduce RBS and start codon ATG in the gene Rrp4
tctagaaataattttgtttaactttaagaaggagatatatatatggctatcgatgcggatatcgcc

SEQ ID NO: 23 - DNA, 167 bp, Artificial Sequence
  End of sequence for the gene Rrp4, promotor and start codon for the gene Rrp41
agatcttaaggtaccctgcaggcttaagtcgaacagaaagtaatcgtattgtacacggccgcaacttcgaaatcaatacgactcactatagggagaccacaacggtttcccatcttagtatattagtttaactttaagaaggagatatacatatggctacgcgtgca

SEQ ID NO: 24 - DNA, 162 bp, Artificial Sequence
  End sequence for the gene Rrp41, promotor e start codon for the gene Rrp42
ggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtcgac

SEQ ID NO: 25 - DNA, 23 bp, Artificial Sequence
  End sequence for the gene Rrp42
ctcgagtaagcggccgcaagctt

SEQ ID NO: 26 - DNA, 783 bp, Artificial Sequence
  Synthetic gene for protein Rrp4, aa 2 until 262
  CDS (1)..(783): aa 2 until 262 of the gene Rrp4
aaacgtatcttcgtacagaatcgtgaactggtggtgccgggtactctgctggcccagggtccttataaaaacggtcgtggcactttccgtgaaggtagccgtatctactctaccgttatcggtctggtggacatcaaaggcaacaccattcgtgtgattcctctggagggtccgtacatcccggaagtaggtgacaacgttatcggtaaaattgtggacgttaagttctctagctgggttgtagacattggtgcgccgtatccggccaacttgaagattcaggacttcactgatgaaaaaattgatttgctgcgtactgaccttcgtaagttctttgatattggtgacattatctacgctaaagtaaaagccatcaccgaagttaacaacattgacctgacgaccaaaggcatgccgtttaacggtggtccgctgaaaggtggtcaaatcgtgaaaattacgccgtctcgtgtgccgcgtgttatcggtcgcggtggcagcatgattaacatgattaaaaaactgaccatgactcgtattattgttggccaaaacggctggatttgggtgtctggcaaaaacgatgcgctggaaaaacttgctattgaggccattctgaaaattgacaaagagtctcatacccgtggtctgactgaccgtattaaagcgctgctgctgtcccgcctgcaagaactgaaagaaaaaggtgttattgaagagattccgaaactggaagaagagccgcagggcgaagatgaagtaaacggcaatgacggtgaagct

SEQ ID NO: 27 - protein, 261 aa, Artificial Sequence
  Synthetic Construct
KRIFVQNRELVVPGTLLAQGPYKNGRGTFREGSRIYSTVIGLVDIKGNTIRVIPLEGPYIPEVGDNVIGKIVDVKFSSWVVDIGAPYPANLKIQDFTDEKIDLLRTDLRKFFDIGDIIYAKVKAITEVNNIDLTTKGMPFNGGPLKGGQIVKITPSRVPRVIGRGGSMINMIKKLTMTRIIVGQNGWIWVSGKNDALEKLAIEAILKIDKESHTRGLTDRIKALLLSRLQELKEKGVIEEIPKLEEEPQGEDEVNGNDGEA

SEQ ID NO: 28 - DNA, 732 bp, Artificial Sequence
  Synthetic gene for the protein Rrp41, aa 3 until 246, double mutant R89T and K91S
  CDS (1)..(732): Synthetic gene for the protein Rrp41, aa 3 until 246, double mutant R89T and K91S
gagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgag

SEQ ID NO: 29 - protein, 244 aa, Artificial Sequence
  Synthetic Construct
EKPEGLKLIDENGRRIDGRKKYELRPIKMEVGVLKNANGSAYIEWGKNKIIAAVYGPRELHPKHLQRPDRAILRVRYNMAPFSVEETKSPGPDRRSIEISKVIKGALEPALILEMFPRTAIDVFIEVLQADAGTRVAGITAASLALADAGIPMRDLVAACAAGKIEGEIVLDLNKEEDNYGEADVPVAIMPLKNDITLLQMDGYLTKDEFIEAVKLAIKGAKAVYQKQREALKEKYLKIAQEVE

SEQ ID NO: 30 - DNA, 819 bp, Artificial Sequence
  Synthetic gene for the Rrp42 protein, aa 2 until 274
  CDS (1)..(819): Rrp42 protein, aa 1 until 274
tctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacag

SEQ ID NO: 31 - protein, 273 aa, Artificial Sequence
  Synthetic Construct
SDNEIVAGIMRDHIINLLKEGKRIDDRGFEDYRPIEIEVGVIEKAEGSALVKLGSTQVLVGIKTSLGEPFPDTPNMGVMTTNVELVPLASPTFEPGPPDERAIELARVIDRGIRESKALNLEKMVIVPGKIVRVVFIDVHVLDHDGNLMDAIGIAAIAALLNARVPKVRYNEETGEVETLDETEPLPVEKIPVPVTFAKIGNILVVDPSLDEELVMDGKITITTDETGHISAVQKSEGGAFKLEEVMYAVETAFKKAEEIRKLILEAVEKAKQ

SEQ ID NO: 32 - DNA, 2752 bp, Artificial Sequence
  Synthetic gene for the three components of the P. abyssi exosome complex
  misc_feature (1)..(6): Restriction site XbaI
  CDS (43)..(858): Rrp4 protein of Pyroccocus abyssi
  misc_feature (49)..(63): Restriction sites ClaI and EcoRV
  promotor (922)..(942): T7 promotor for the gene of Rrp41
  misc_feature (922)..(942): Site Bgl II, Stop codon, restriction sites KpnI and PstI
  misc_feature (999)..(1013): Restriction sited NdeI and MluI
  CDS (1002)..(1757): R89T and K91S double mutant protein of Rrp41 from Pyrococcus abyssi
  misc_feature (1749)..(1769): Site BamHI, Stop codon, restriction sites SpeI and EcoRI
  promotor (1822)..(1842): T7 promotor for the gene of Rrp42
  misc_feature (1897)..(1910): Restriction sites NcoI and SaII
  CDS (1899)..(2738): Rrp42 protein of Pyrococcus abyssi
  misc_feature (2730)..(2752): Site XhoI, Stop codon, restriction sites NotI and HindIII
tctagaaataattttgtttaactttaagaaggagatatatatatggctatcgatgcggatatcgccaaacgtatcttcgtacagaatcgtgaactggtggtgccgggtactctgctggcccagggtccttataaaaacggtcgtggcactttccgtgaaggtagccgtatctactctaccgttatcggtctggtggacatcaaaggcaacaccattcgtgtgattcctctggagggtccgtacatcccggaagtaggtgacaacgttatcggtaaaattgtggacgttaagttctctagctgggttgtagacattggtgcgccgtatccggccaacttgaagattcaggacttcactgatgaaaaaattgatttgctgcgtactgaccttcgtaagttctttgatattggtgacattatctacgctaaagtaaaagccatcaccgaagttaacaacattgacctgacgaccaaaggcatgccgtttaacggtggtccgctgaaaggtggtcaaatcgtgaaaattacgccgtctcgtgtgccgcgtgttatcggtcgcggtggcagcatgattaacatgattaaaaaactgaccatgactcgtattattgttggccaaaacggctggatttgggtgtctggcaaaaacgatgcgctggaaaaacttgctattgaggccattctgaaaattgacaaagagtctcatacccgtggtctgactgaccgtattaaagcgctgctgctgtcccgcctgcaagaactgaaagaaaaaggtgttattgaagagattccgaaactggaagaagagccgcagggcgaagatgaagtaaacggcaatgacggtgaagctagatcttaaggtaccctgcaggcttaagtcgaacagaaagtaatcgtattgtacacggccgcaacttcgaaatcaatacgactcactatagggagaccacaacggtttcccatcttagtatattagtttaactttaagaaggagatatacatatggctacgcgtgcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtcgactctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagtaagcggccgcaagctt

SEQ ID NO: 33 - protein, 271 aa, Artificial Sequence
  Synthetic Construct
MAIDADIAKRIFVQNRELVVPGTLLAQGPYKNGRGTFREGSRIYSTVIGLVDIKGNTIRVIPLEGPYIPEVGDNVIGKIVDVKFSSWVVDIGAPYPANLKIQDFTDEKIDLLRTDLRKFFDIGDIIYAKVKAITEVNNIDLTTKGMPFNGGPLKGGQIVKITPSRVPRVIGRGGSMINMIKKLTMTRIIVGQNGWIWVSGKNDALEKLAIEAILKIDKESHTRGLTDRIKALLLSRLQELKEKGVIEEIPKLEEEPQGEDEVNGNDGEARS

SEQ ID NO: 34 - protein, 251 aa, Artificial Sequence
  Synthetic Construct
MATRAEKPEGLKLIDENGRRIDGRKKYELRPIKMEVGVLKNANGSAYIEWGKNKIIAAVYGPRELHPKHLQRPDRAILRVRYNMAPFSVEETKSPGPDRRSIEISKVIKGALEPALILEMFPRTAIDVFIEVLQADAGTRVAGITAASLALADAGIPMRDLVAACAAGKIEGEIVLDLNKEEDNYGEADVPVAIMPLKNDITLLQMDGYLTKDEFIEAVKLAIKGAKAVYQKQREALKEKYLKIAQEVEGS

SEQ ID NO: 35 - protein, 279 aa, Artificial Sequence
  Synthetic Construct
MGVDSDNEIVAGIMRDHIINLLKEGKRIDDRGFEDYRPIEIEVGVIEKAEGSALVKLGSTQVLVGIKTSLGEPFPDTPNMGVMTTNVELVPLASPTFEPGPPDERAIELARVIDRGIRESKALNLEKMVIVPGKIVRVVFIDVHVLDHDGNLMDAIGIAAIAALLNARVPKVRYNEETGEVETLDETEPLPVEKIPVPVTFAKIGNILVVDPSLDEELVMDGKITITTDETGHISAVQKSEGGAFKLEEVMYAVETAFKKAEEIRKLILEAVEKAKQLE

SEQ ID NO: 36 - DNA, 5754 bp, Artificial Sequence
  pMRKA-EXO plasmid for the expression of recombinant proteins in fusion with proteins of Pyrococcus abyssi exosome
gatctcgatcccgcgaaattaatacgactcactatagggagaccacaacggtttccctctagaaataattttgtttaactttaagaaggagatatatatatggctatcgatgcggatatcgccaaacgtatcttcgtacagaatcgtgaactggtggtgccgggtactctgctggcccagggtccttataaaaacggtcgtggcactttccgtgaaggtagccgtatctactctaccgttatcggtctggtggacatcaaaggcaacaccattcgtgtgattcctctggagggtccgtacatcccggaagtaggtgacaacgttatcggtaaaattgtggacgttaagttctctagctgggttgtagacattggtgcgccgtatccggccaacttgaagattcaggacttcactgatgaaaaaattgatttgctgcgtactgaccttcgtaagttctttgatattggtgacattatctacgctaaagtaaaagccatcaccgaagttaacaacattgacctgacgaccaaaggcatgccgtttaacggtggtccgctgaaaggtggtcaaatcgtgaaaattacgccgtctcgtgtgccgcgtgttatcggtcgcggtggcagcatgattaacatgattaaaaaactgaccatgactcgtattattgttggccaaaacggctggatttgggtgtctggcaaaaacgatgcgctggaaaaacttgctattgaggccattctgaaaattgacaaagagtctcatacccgtggtctgactgaccgtattaaagcgctgctgctgtcccgcctgcaagaactgaaagaaaaaggtgttattgaagagattccgaaactggaagaagagccgcagggcgaagatgaagtaaacggcaatgacggtgaagctagatcttaaggtaccctgcaggcttaagtcgaacagaaagtaatcgtattgtacacggccgcaacttcgaaatcaatacgactcactatagggagaccacaacggtttcccatcttagtatattagtttaactttaagaaggagatatacatatggctacgcgtgcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtcgactctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagtaagcggccgcaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 37 - DNA, 4795 bp, Artificial Sequence
  Expression vector by fusion antigen with Rrp41, Rrp42 proteins of Pyrococcus abyssi exosome
gatctcgatcccgcgaaattaatacgactcactatagggagaccacaacggtttccctctagaaataattttgtttaactttaagaaggagatatacatatggctacgcgtgcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtcgactctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagtaagcggccgcaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 38 - DNA, 29 bp, Artificial Sequence
  Foward primer for PCR amplification of Rrp42 gene
accatatgggttctgataatgaaatcgtg

SEQ ID NO: 39 - DNA, 47 bp, Artificial Sequence
  Reverse primer for PCR amplification of Rrp42 gene
aactgcagggatccctcgagaccaccctgtttggccttctcaacagc

SEQ ID NO: 40 - DNA, 3917 bp, Artificial Sequence
  Expression vector for recombinant fusion proteins by fusion with Rrp42 of P. abyssi exosome
gatctcgatcccgcgaaattaatacgactcactatagggagaccacaacggtttccctctagaaataattttgtttaactttaagaaggagatatacatatgggttctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagggtggtctcgagggatccctgcagctggtaccatggaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 41 - DNA, 368 bp, Artificial Sequence
  coding artificial sequence of z-z domain of protein A of S. aureus
ccatgggtgtggataacaaatttaacaaagaacagcagaatgcgttttacgaaattctgcacctgccgaaccttaacgaagaacagcgtaacgctttcattcagtctctgaaagatgacccttctcagtctgcgaacctgctggctgaagctaaaaaactgaacgacgctcaggctcctaaagttgacaacaaattcaacaaagaacagcagaacgctttctacgaaatcttgcatctgccgaacctgaacgaagaacagcgcaacgctttcatccagtccctgaaagacgacccttcccagtctgctaacctgctggcggaagcgaaaaaactgaacgatgctcaggcgcctaaagtgggcgtcgac

SEQ ID NO: 42 - protein, 122 aa, Artificial Sequence
  Synthetic construct
MGVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGVD

SEQ ID NO: 43 - DNA, 5148 bp, Artificial Sequence
  Expression vector by fusion of Rrp41 and ZZ- Rrp42 proteins
gatctcgatcccgcgaaattaatacgactcactatagggagaccacaacggtttccctctagaataattttgtttaactttaagaaggagatatacatatggctacgcgtgcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtggataacaaatttaacaaagaacagcagaatgcgttttacgaaattctgcacctgccgaaccttaacgaagaacagcgtaacgctttcattcagtctctgaaagatgacccttctcagtctgcgaacctgctggctgaagctaaaaaactgaacgacgctcaggctcctaaagttgacaacaaattcaacaaagaacagcagaacgctttctacgaaatcttgcatctgccgaacctgaacgaagaacagcgcaacgctttcatccagtccctgaaagacgacccttcccagtctgctaacctgctggcggaagcgaaaaaactgaacgatgctcaggcgcctaaagtgggcgtcgactctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagtaagcggccgcaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 44 - protein, 405 aa, Artificial Sequence
  Synthetic construct
LSIRRRYTMGVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVGVDSDNEIVAGIMRDHIINLLKEGKRIDDRGFEDYRPIEIEVGVIEKAEGSALVKLGSTQVLVGIKTSLGEPFPDTPNMGVMTTNVELVPLASPTFEPGPPDERAIELARVIDRGIRESKALNLEKMVIVPGKIVRVVFIDVHVLDHDGNLMDAIGIAAIAALLNARVPKVRYNEETGEVETLDETEPLPVEKIPVPVTFAKIGNILVVDPSLDEELVMDGKITITTDETGHISAVQKSEGGAFKLEEVMYAVETAFKKAEEIRKLILEAVEKAKQLE

SEQ ID NO: 45 - DNA, 5088 bp, Artificial Sequence
  Expression vector by fusion of recombinant proteins with Rrp41 and ZZ-Rrp42 of P. abyssi exosome
  CDS (966)..(2189): Z-Z Rrp42 Protein
gatctcgatcccgcgaaattaatacgactcactatagggagaccacaacggtttccctctagaataattttgtttaactttaagaaggagatatacatatggctacgcgtgcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcctaaactagtgaattcgcttaagtcgaacagaaagtaatcgtattgtacacggccgcataatcgaaattaatacgactcactatagggagaccacaacggtttcccatcttagtatattagttaagtataagaaggagatataccatgggtgtggataacaaatttaacaaagaacagcagaatgcgttttacgaaattctgcacctgccgaaccttaacgaagaacagcgtaacgctttcattcagtctctgaaagatgacccttctcagtctgcgaacctgctggctgaagctaaaaaactgaacgacgctcaggctcctaaagttgacaacaaattcaacaaagaacagcagaacgctttctacgaaatcttgcatctgccgaacctgaacgaagaacagcgcaacgctttcatccagtccctgaaagacgacccttcccagtctgctaacctgctggcggaagcgaaaaaactgaacgatgctcaggcgcctaaagtgggcgtcgactctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagtaagcggccgcaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattaggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacgtcgagttatggacagcaagcgaaccggaattgccagctggggcgccctctggtaaggttgggaagccctgcaaagtaaactggatggctttcttgccgccaaggatctgatggcgcaggggatcaagatttgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaattctagtgcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcagtcgagtttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtctagtacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag